(19) United States
(12) Patent Application Publication
(10) Pub. No.: US 2007/0148676 A1
(43) Pub. Date: Jun. 28, 2007

(54) Methods for detecting gene expression in peripheral blood cells and uses thereof

(76) Inventors: Sylvia G. Kachalsky, Gan Yavne (IL); Guy Horev, Rehovot (IL)

Correspondence Address: WINSTON & STRAWN LLP;PATENT DEPARTMENT, 1700 K STREET, N.W., WASHINGTON, DC, 20006, US

(21) Appl. No.: 11/633,063

(22) Filed: Dec. 1, 2006

Related U.S. Application Data
(63) Continuation of application No. PCT/IL05/00590, filed on Jun. 5, 2005.
(60) Provisional application No. 60/576,599, filed on Jun. 4, 2004.

(51) Int. Cl. C12Q 1/68

(52) U.S. Cl. 435/6

(57) ABSTRACT

The present invention relates to methods of identifying biomarkers for disease by measuring gene expression levels in subpopulations of blood cells obtained from subjects of closed populations. Particularly, the present invention relates to methods of diagnosing, monitoring and prognosing diseases by determining expression levels of disease-specific genes.

### CROSS-REFERENCE TO RELATED APPLICATIONS

This application is a continuation of International application PCT/IL2005/000590 Filed Jun. 5, 2005, which claims the benefit of provisional application 60/576,599 filed Jun. 4, 2004, the entire content of each of which is expressly incorporated herein by reference thereto.

### FIELD OF THE INVENTION

The present invention relates to methods of identifying biomarkers for a disease, which comprise measuring gene expression levels in subpopulations of blood cells obtained from subjects of closed populations. Particularly, the present invention relates to methods of diagnosing and prognosing diseases comprising determining expression levels of disease-specific genes.

### BACKGROUND OF THE INVENTION

The functional changes in the immune system enable it to specifically react to any given challenge to the healthy steady state of the body. The immune system and its constantly circulating white blood cells or leukocytes, are in constant interaction with the different tissues in the body. Given any patho-physiological stimulus, peripheral circulating leukocytes detect and specifically react based on their ability to measure the normal or steady state body situation. This specific reaction can be measured by means of functional genomics and proteomics. In schizophrenia, for example, the expression level of dopamine receptors, which are known to be associated with a number of neuropathological disorders, has been shown to be higher in lymphocytes of schizophrenia patients than in healthy individuals (Hani, T. et al., Proc. Natl. Acad. Sci. USA 98: 625-628, 2001). Positive correlation between the expression of a gene and a particular disease has been also documented for other diseases such as heart failure disease and hypertension.

While many studies have been aimed at identifying changes in expression of individual genes, there is a growing awareness that many diseases affect the expression of a large number of genes. In cases where the genes participate in the same signaling pathway, the involvement of these genes may be expected. However, in cases where the genes participate in separate signaling pathways, the involvement of these genes is totally unexpected. DNA-based arrays can provide a simple way to explore simultaneously and accurately the expression of a large number of genes.

cDNA-based arrays have been used to profile complex diseases and discover novel disease-related genes. In rheumatoid arthritis, the expression patterns of selected genes in tissue samples as detected by cDNA-based arrays have been shown to be different from the expression patterns obtained from tissue samples of individuals having other inflammatory diseases (Heller R. A., et al. Proc. Natl. Acad. Sci. USA 94: 2150-2155, 1997). Variations in gene expression in peripheral blood mononuclear cells have been documented in atopy and asthma and a composite atopy gene expression (CAGE) score was determined by using 10 genes dysregulated in atopic individuals according to a specific algorithm (Brutsche, M. H., et al., J. Allergy Clin. Immunol. 109: 271-273, 2002). Gene expression profiles for myeloma and cardiovascular diseases have been also documented in leukocytes (see, for example, Claudio, J. O., et al., Blood 100: 2175-2186, 2002). Recently, application of genome-wide expression analysis in leukocytes to study human diseases has been documented (Cobb, J. P., et. al., Proc. Natl. Acad. Sci. USA 102: 4801-4806, 2005).

International Patent Application WO 2004/112589 relates to the identification of biomarkers in blood samples for different diseases. The methods for identifying a biomarker for a disease according to WO 2004/112589 comprise determining the level of one or more RNA transcripts expressed in blood obtained from one or more individuals having a disease and comparing the level of each of said one or more RNA transcripts with the level of each of said one or more RNA transcripts in blood obtained from one or more individuals not having the disease, wherein the RNA transcripts which display differing levels are identified as biomarkers. WO 2004/112589 further provides methods for diagnosing a condition in an individual comprising determining the level of the gene transcripts, which correspond to the biomarkers of the disease and kits comprising said biomarkers. WO 2004/112589 further discloses that when comparing between two populations of individuals having and not having a particular disease in order to identify biomarkers of a disease, such populations preferably share at least one phenotype in common. Examples of phenotypes that can be in common in such populations include similar age, sex and body mass index (BMI). There is no indication that the individuals belong to a closed or founder population.

U.S. Patent Application No. 2005/0042630 relates to methods of identifying markers for asthma. The methods for identifying markers for asthma according to U.S. Patent Application No. 2005/0042630 comprise determining the level of one or more gene transcripts in blood obtained from one or more individuals having asthma, wherein each of said one or more transcripts is expressed by a gene that is a candidate marker for asthma and comparing the level of each of said one or more gene transcripts with the level of each of said one or more genes transcripts in blood obtained from one or more individuals not having asthma, wherein those compared transcripts which display differing levels are identified as being markers for asthma. U.S. Patent Application No. 2005/0042630 further provides methods for diagnosing or prognosing asthma in an individual comprising determining the level of the gene transcripts, which correspond to asthma. U.S. Patent Application No. 2005/0042630 claims isolated nucleic acid molecules that correspond to two or more of the asthma markers, an array consisting essentially of said nucleic acid molecules and a kit for diagnosing or prognosing asthma. U.S. Patent Application No. 2005/0042630 makes use of whole blood samples, which includes all the types of blood cells. It is indicated explicitly in U.S. Patent Application No. 2005/0042630 that use of whole blood is of great advantage, as purifying blood cells is costly and time consuming.

Founder populations offer many advantages for mapping genetic traits, particularly complex traits that are likely to be genetically heterogeneous. It is well established that since founder populations are relatively genetically homogenous, the molecular genetic mechanism underlying genetically heterogeneous diseases is easier to dissect in founder populations than in mixed populations. Indeed, Ober, C., et al., have conducted a genome-wide screen in asthma patients of Hutterites, a religious isolate of European ancestry, and identified twelve markers that showed possible linkage to asthma (Ober, C., et al, Hum. Mol. Genet. 7: 1393-1398, 1998). Laitinen, T. et al., have carried out a genome-wide scan for susceptibility loci in asthma individuals of a founder population in Finland, and found evidence for linkage in chromosome 7p14-p15. Thus, although genotype analysis and linkage disequilibrium studies in founder populations were found to be instrumental in finding susceptible disease loci in each closed population, they failed in determining diagnostic tools in the general population. Complex polygenic diseases were found to be characterized by different mutated loci in different populations making the generalization of one loci-one disease impossible.

There is an unmet need for accurate and reliable methods of diagnosing a disease in a subject, which methods neither comprise detecting susceptibility loci for the disease nor comprise determining the level of gene transcripts, which correspond to candidate markers of the disease as identified in open populations.

### SUMMARY OF THE INVENTION

The present invention relates to methods of diagnosis, prognosis and monitoring of a disease in a subject comprising determining the level of gene expression in isolated blood cells obtained from the subject.

It is now disclosed that isolated and purified blood cells enable achieving accurate and reproducible gene expression profiles of a disease as compared to those obtained with whole blood samples. Though the isolation of specific blood cells constitutes an additional step in the process of obtaining a gene expression profile from a blood sample, the significant differences in the levels of gene transcripts obtained from isolated blood cell samples as compared to whole blood samples substantiates the necessity of this step in order to provide highly reproducible gene expression profiles for a particular disease.

It is further disclosed that the use of a closed or founder population, which a priori has a lower genetic variation than that of an open population, enables obtaining a precise and reproducible gene expression profile of a particular disease. Thus, the use of isolated blood cells of subjects that belong to a closed or founder population is highly advantageous as it provides a reliable and consistent gene expression profile for a particular disease.

It is further disclosed that a disease-specific profile of gene expression is useful in diagnosis, prognosis, and monitoring a disease in isolated blood cell samples of a subject.

According to the first aspect, the present invention provides a method of identifying at least one biomarker for a disease comprising the steps of: a) determining the level of at least one gene transcript in a subpopulation of blood cells obtained from at least one subject having the disease, the at least one subject having the disease being a member of a closed population; and b) comparing the level of the at least one gene transcript from step a) with the level of said at least one gene transcript in the subpopulation of blood cells obtained from at least one subject not having the disease, the at least one subject not having the disease being a member of the closed population, wherein a gene transcript which displays significantly differing levels in the comparison of step b) is identified as being a biomarker for said disease.

According to some embodiments, the method of identifying at least one biomarker for a disease further comprises the following steps: c) determining the level of said at least one gene transcript in said subpopulation of blood cells obtained from at least one subject having the disease, the at least one subject having the disease being a member of an open population; and d) comparing the level of the at least one gene transcript from step c) with the level of said at least one gene transcript in the subpopulation of blood cells obtained from at least one subject not having the disease, the at least one subject not having the disease being a member of an open population, wherein a gene transcript which displays corresponding changes in levels of expression in the comparisons of steps b) and d) is identified as being a biomarker for said disease.

According to additional embodiments, the corresponding changes in levels of expression in the comparisons of steps b) and d) are increasing levels. According to other embodiments, the corresponding changes in levels of expression in the comparisons of steps b) and d) are decreasing levels.

According to some embodiments, the at least one biomarker for a disease is a plurality of biomarkers. According to additional embodiments, the plurality of biomarkers comprises at least 5 biomarkers. According to further embodiments, the plurality of biomarkers comprises at least 10 biomarkers. According to further embodiments, the plurality of biomarkers comprises at least 100 biomarkers. According to further embodiments, the plurality of biomarkers comprises at least 200 biomarkers. According to further embodiments, the plurality of biomarkers comprises at least 500 biomarkers.

According to some embodiments, the closed or founder population is selected from the group consisting of Quebecois, Icelandic, Dutch, East Central Finnish, North American Hutterites, Sicilian, Israel Arabic, Bedouin, Charkese, Ashkenazi Jewish, Cochin Jewish populations, Ethiopian Jewish, Iraquian Jewish, Yemenite Jewish and Iranian Jewish.

According to some embodiments, the subpopulation of blood cells is peripheral white blood cells or leukocytes. According to additional embodiments, the subpopulation of blood cells is selected from the group consisting of monocytes, lymphocytes, neutrophils, eosinophils, and basophils.

According to additional embodiments, the disease for which biomarkers can be identified by the methods of the present invention is selected from the group consisting of cardiovascular disorders, immune disorders, autoimmune diseases, respiratory diseases, endocrine disorders, neurological disorders, muscular disorders, metabolic disorders, mood disorders, and cellular proliferative disorders.

According to an exemplary embodiment, the disease for which biomarkers can be identified is asthma. According to some embodiments, the biomarkers for asthma are selected from the group consisting of SEQ ID NOs:1-783 and complements thereof. According to additional embodiments, the biomarkers for asthma are selected from the group consisting of the sequences listed in Table 4 herein below and complements thereof. It is to be understood that the levels of gene transcripts corresponding to the biomarkers listed in Table 4 herein below were significantly different in subjects having asthma as compared to subjects not having asthma. Additionally, it is to be understood that gene transcripts identified according to the methods of the present invention as being biomarkers of a disease can be translated to polypeptides or proteins. Accordingly, the polypeptides or proteins can be identified as being biomarkers according to the principles of the present invention.

According to a further aspect, the present invention provides a plurality of isolated nucleic acid molecules corresponding to one or more of the biomarkers, identified by the methods of the present invention, or complements thereof.

According to another aspect, the present invention provides an array comprising a plurality of isolated nucleic acid molecules, wherein the isolated nucleic acid molecules corresponding to one or more of the biomarkers, identified by the methods of the present invention, or complements thereof.

According to another aspect, the present invention provides a method of diagnosing, monitoring or prognosing a disease in a subject comprising the steps of:
a) determining the level of at least one gene transcript in a subpopulation of blood cells obtained from the subject, wherein the at least one gene transcript corresponds to a biomarker, the biomarker having been determined by the steps of:
   i) determining the level of at least one gene transcript in a subpopulation of blood cells obtained from at least one subject having the disease, the at least one subject having the disease being a member of a closed population; and
   ii) comparing the level of the at least one gene transcript from step i) with the level of said at least one gene transcript in the subpopulation of blood cells obtained from at least one subject not having the disease, the at least one subject not having the disease being a member of the closed population, wherein a gene transcript which displays significantly differing levels in the comparison of step i) is identified as being a biomarker for said disease;
b) comparing the level of said at least one gene transcript of step a) with the level of said at least one gene transcript in a reference gene transcript profile, thereby determining the status of the disease in said subject.

According to some embodiments, the method of diagnosing, monitoring or prognosing a disease in a subject further comprising the steps of:
a) determining the level of at least one gene transcript in a subpopulation of blood cells obtained from the subject, wherein the at least one gene transcript corresponds to a biomarker, the biomarker having been determined by the steps of:
   i) determining the level of at least one gene transcript in a subpopulation of blood cells obtained from at least one subject having the disease, the at least one subject having the disease being a member of a closed population;
   ii) comparing the level of the at least one gene transcript from step i) with the level of said at least one gene transcript in the subpopulation of blood cells obtained from at least one subject not having the disease, the at least one subject not having the disease being a member of the closed population, wherein a gene transcript which displays significantly differing levels in the comparison of step i) is identified as being a biomarker for said disease;
   iii) determining the level of said at least one gene transcript in said subpopulation of blood cells obtained from at least one subject having the disease, the at least one subject having the disease being a member of an open population; and
   iv) comparing the level of the at least one gene transcript from step iii) with the level of said at least one gene transcript in the subpopulation of blood cells obtained from at least one subject not having the disease, the at least one subject not having the disease being a member of an open population, wherein a gene transcript which displays corresponding changes in levels of expression in the comparisons of steps ii) and iv) is identified as being a biomarker for said disease;
b) comparing the level of said at least one gene transcript of step a) with the level of said at least one gene transcript in a reference gene transcript profile, thereby determining the status of the disease in said subject.

According to some embodiments, the step of determining the level of the at least one gene transcript comprises determining the expression level of the gene. According to additional embodiments, the step of determining the level of the at least one gene transcript comprises determining the expression level of the polypeptide gene transcript.

According to some embodiments, the subpopulation of blood cells is peripheral white blood cells or leukocytes. According to other embodiments, the subpopulation of blood cells is selected from the group consisting of monocytes, lymphocytes, neutrophils, eosinophils, and basophils.

According to some embodiments, the biomarker for diagnosing, monitoring or prognosing the disease is a plurality of biomarkers.

According to additional embodiments, the disease that can be diagnosed, monitored or prognosed is selected from the group consisting of cardiovascular disorders, immune disorders, autoimmune diseases, respiratory diseases, endocrine disorders, neurological disorders, muscular disorders, metabolic disorders, mood disorders, and cellular proliferative disorders.

According to an exemplary embodiment, the disease is asthma. According to other embodiments, the biomarkers for asthma are selected from the group consisting of SEQ ID NOs:1-783 and complements thereof. According to some preferred embodiments, the biomarkers of asthma are selected from the group consisting of the sequences listed in Table 4 herein below and complements thereof.

According to some embodiments, the step of determining the level of at least one gene transcript comprises quantitative or semi-quantitative methods. According to some embodiments, the quantitative or semi-quantitative methods measure levels of nucleic acid. According to alternative embodiments, the quantitative or semi-quantitative methods measure levels of polypeptides. According to an exemplary embodiment, the step of determining the level of at least one gene transcript comprises microarray hybridization.

According to additional embodiments, the microarray hybridization comprises hybridizing a plurality of first isolated nucleic acid molecules to an array comprising a second plurality of isolated nucleic acid molecules. The first isolated nucleic acid molecules are selected from the group consisting of RNA, DNA, cDNA and PCR products. The second isolated nucleic acid molecules are selected from the group consisting of RNA, DNA, cDNA, PCR products, oligonucleotides and ESTs. According to one exemplary embodiment, the first isolated nucleic acid molecules are cDNAs and the second isolated nucleic acid molecules are ESTs or oligonucleotides, the cDNA and ESTs or oligonucleotides capable of hybridizing to each other. According to some embodiments, the second isolated nucleic acid molecules correspond to one or more biomarkers, identified by the methods of the present invention, or complements thereof.

These and other embodiments of the present invention will be better understood in relation to the figures, description, examples and claims that follow.

### BRIEF DESCRIPTION OF THE FIGURES

FIG. 1 shows a dendogram of all the genes of the asthma and non-asthma individuals in the Cochin population using human array A. The horizontal axis identifies individuals as asthmatic and non-asthmatic and the vertical axis represents the statistical variance between the samples (0.10 means 5%, 0.20 means 10%, etc.).

FIG. 2 shows a dendogram of all the genes of the asthma and non-asthma individuals in the Cochin population using human array B. The horizontal axis identifies individuals as asthmatic and non-asthmatic and the vertical axis represents the statistical variance between the samples (0.10 means 5%, 0.20 means 10%, etc.).

FIG. 3 shows a dendogram of all the genes comprising the “asthma primary gene expression profile” of the asthma and non-asthma individuals in the Cochin population using human array A. The horizontal axis identifies individuals as asthmatic and non-asthmatic and the vertical axis represents the statistical variance between the samples (0.10 means 5%, 0.20 means 10%, etc.).

FIG. 4 shows a dendogram of all the genes comprising the “asthma primary gene expression profile” of the asthma and non-asthma individuals in the Cochin population using human array B. The horizontal axis identifies individuals as asthmatic and non-asthmatic and the vertical axis represents the statistical variance between the samples (0.10 means 5%, 0.20 means 10%, etc.).

### DETAILED DESCRIPTION OF THE INVENTION

The present invention provides methods of identifying biomarkers, which correspond to one or more gene transcripts that are differentially expressed in peripheral blood cells of a subject having a disease or in a diseased subject undergoing a treatment. Also disclosed herein isolated nucleic acid molecules corresponding to said biomarkers as well as methods of diagnosing a disease using the biomarkers. Arrays comprising the biomarkers or complementary nucleic acid molecules thereof are disclosed.

According to the present invention, a blood sample is collected from one or more subjects, peripheral blood cells are purified, and RNA is isolated from the cells. According to some embodiments, the peripheral blood cells are peripheral white blood cells or leukocytes. As white blood cells or leukocytes include different cell types such as lymphocytes, monocytes, neutrophils, eosinophils and basophils, the present invention encompasses one or more of the blood cell subpopulations.

Biomarkers are identified by measuring the level of one or more gene transcripts or a synthetic nucleic acid copy (cDNA, cRNA, etc.) thereof from one or more subjects having a disease and comparing the level of the one or more gene transcripts to that of one or more subjects not having the disease and/or normal healthy subjects. In some embodiments, the level of one or more gene transcripts is determined by determining the level of an RNA species. In one embodiment, mass spectrometry can be used to quantify the level of one or more gene transcripts. In a preferred embodiment, the level of one or more gene transcripts is determined using microarray analysis. Other methods to quantify gene transcripts include, for example, quantitative RT-PCR and conventional molecular biology and recombinant DNA techniques aiming at quantitatively or semi-quantitatively measure one or more species of gene transcripts (see, for example, Sambrook, Fritsch and Maniatis, “Molecular Cloning: A Laboratory Manual (1982); “DNA Cloning: A Practical Approach,” Volumes I and II (D. N. Glover ed. 1985); B. Perbal, “A Practical Guide To Molecular Cloning” (1984)).

Other methods to quantify or semi-quantify a gene transcript include determining the level of polypeptides or proteins in a blood sample. Methods for measuring levels of a polypeptide or protein are well known in the art and include, but are not limited to, enzyme linked immunosorbent assay (ELISA), western blotting, protein or antibody based biosensors and mass spectrometry.

According to some embodiments of the invention, levels of one or more species of gene transcripts from a blood cell sample of at least one subject having a disease, wherein the subject being a member of a closed or founder population, are compared to levels of the one or more species of gene transcripts from a blood cell sample of a subject not having the disease, wherein the subject not having the disease being a member of said closed or founder population, so as to identify biomarkers, which are able to differentiate between the two populations. According to other embodiments, blood cell samples of at least two subjects from each of said populations are compared. According to additional embodiments, blood samples of at least 5 subjects from each of said populations are compared.

The identified biomarkers can be used for diagnosing, monitoring or prognosing a disease in a subject.

Definitions

A “cDNA” is defined as a complementary DNA and is a product of a reverse transcription reaction from an mRNA template. “RT-PCR” refers to reverse transcription polymerase chain reaction and results in production of cDNAs that are complementary to the mRNA template(s). RT-PCR includes quantitative real time RT-PCR, which uses a labeling means to determine the level of mRNA transcription.

The term “oligonucleotide” is defined as a molecule comprised of two or more deoxyribonucleotides and/or ribonucleotides, preferably more than three. Its exact size will depend upon many factors, which, in turn, depend upon the ultimate function and use of the oligonucleotide. The upper limit may be 15, 20, 25, 30, 40, 50, 60 or 70 nucleotides in length.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent.

The term “subject” refers to human subjects and non-human subjects.

As used herein, “determining” refers to detecting the presence of or measuring the level or concentration of a gene expression product, for example cDNA or RNA by any method known to those of skill in the art or taught in numerous texts and laboratory manuals (see, for example, Ausubel et al. Short Protocols in Molecular Biology (1995) 3rd Ed. John Wiley & Sons, Inc.). For example, methods of detection include, but are not limited to, RNA fingerprinting, Northern blotting, polymerase chain reaction, ligase chain reaction, strand displacement amplification, transcription based amplification, and other methods as known in the art.

As used herein, a disease of the invention includes, but is not limited to, blood disorders, blood lipid diseases, autoimmune diseases, arthritis (including osteoarthritis, rheumatoid arthritis, lupus, allergies, juvenile rheumatoid arthritis and the like), bone or joint disorders, cardiovascular disorders (including heart failure, congenital heart disease; rheumatic fever, valvular heart disease; corpulmonale, cardiomyopathy, myocarditis, pericardial disease; vascular diseases such as atherosclerosis, acute myocardial infarction, ischemic heart disease and the like), obesity, respiratory diseases (including asthma, pneumonitis, pneumonia, pulmonary infections, lung disease, bronchiectasis, tuberculosis, cystic fibrosis, interstitial lung disease, chronic bronchitis emphysema, pulmonary hypertension, pulmonary thromboembolism, acute respiratory distress syndrome and the like), hyperlipidemias, endocrine disorders, immune disorders, infectious diseases, neurological disorders (including migraines, seizures, epilepsy, cerebrovascular diseases, Alzheimer's, dementia, Parkinson's, ataxic disorders, motor neuron diseases, cranial nerve disorders, spinal cord disorders, meningitis and the like) including neurodegenerative and/or neuropsychiatric diseases and mood disorders (including schizophrenia, anxiety, bipolar disorder; manic depression and the like), skin disorders, kidney diseases, scleroderma, strokes, hereditary hemorrhage telangiectasia, diabetes, disorders associated with diabetes (e.g., PVD), hypertension, Gaucher's disease, cystic fibrosis, sickle cell anemia, liver disease, pancreatic disease, eye, ear, nose and/or throat disease, diseases affecting the reproductive organs, gastrointestinal diseases (including diseases of the colon, diseases of the spleen, appendix, gall bladder, and others) and the like.

According to some embodiments of the invention, a disease refers to an immune disorder, such as those associated with over expression of a gene or expression of a mutant gene (e.g., autoimmune diseases, such as diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scieroderma, vaginitis, proctitis, drug eruptions, leprosy reversal reactions, erythema nodosum leprosum, autoimrnune uveitis, allergic encephalomyelitis, acute necrotizing hemorrhagic encephalopathy, idiopathic bilateral progressive sensorineural hearing, loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, and interstitial lung fibrosis), graft-versus-host disease, cases of transplantation, and allergy.

According to additional embodiments, a disease of the invention is a cellular proliferative and/or differentiative disorder that includes, but is not limited to, cancer e.g., carcinoma, sarcoma or other metastatic disorders and the like. As used herein, the term “cancer” refers to cells having the capacity for autonomous growth, i.e., an abnormal state of condition characterized by rapidly proliferating cell growth. “Cancer” is meant to include all types of cancerous growths or oncogenic processes, metastatic tissues or malignantly transformed cells, tissues, or organs, irrespective of histopathologic type or stage of invasiveness. Examples of cancers include but are nor limited to solid tumors and leukemias, including: apudoma, choristoma, branchioma, malignant carcinoid syndrome, carcinoid heart disease, carcinoma (e.g., Walker, basal cell, basosquamous, Brown-Pearce, ductal, Ehrlich tumor, non-small cell lung, oat cell, papillary, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adeno-carcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hernangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neoplasms (e.g., bone, breast, digestive system, colorectal, liver, pancreatic, pituitary, testicular, orbital, head and neck, central nervous system, acoustic, pelvic respiratory tract, and urogenital), neurofibromatosis, and cervical dysplasia, and other conditions in which cells have become immortalized or transformed.

As defined herein, a “microarray” refers to a plurality of isolated nucleic acid molecules or polynucleotide probes attached to a support where each of the nucleic acid molecules or polynucleotide probes is attached to a support in unique pre-selected region. According to one embodiment, the nucleic acid molecule or polynucleotide probe attached to the support is DNA. In other embodiment, the nucleic acid or polynucleotide probe attached to the support is cDNA. The term “nucleic acid” is interchangeable with the term “polynucleotide”. The term “polynucleotide” refers to a chain of nucleotides. Preferably, the chain has from about 20 to 10,000 nucleotides, more preferably from about 150 to 3,500 nucleotides. The term “probe” refers to a polynucleotide sequence capable of hybridizing with a gene transcript or complement thereof to form a polynucleotide probe/gene transcript complex.

As used herein, a “closed” or “founder” population refers to a population of subjects characterized by a close genetic relationship. A closed population can be further characterized by elevated incidence of certain hereditary disorders and/or a higher prevalence of mutations than in an open or mixed population. Examples of closed or founder populations include, but are not limited to, populations of Quebec, Netherland, Iceland, East Central Finland (Kainuu province), Amish, Newfoundland, Israel Bedouins, Druze, Charkese, Hutterites of North America, Israeli Jewish subpopulation including, but not limited to, the Ethiopian, Iraqi, Yemenite, Ashkenazi, Iranian and Cochin Jewish subpopulations.

The term “gene” includes a region that can be transcribed into RNA, as the invention contemplates detection of RNA or equivalents thereof, for example, cDNA and cRNA. A gene of the invention includes, but is not limited to, genes specific for or involved in a particular biological process and/or indicative of a biological process, such as apoptosis, differentiation, stress response, aging, proliferation, etc.; cellular mechanism genes, e.g., cell-cycle, signal transduction, metabolism of toxic compounds, and the like; disease associated genes, e.g., genes involved in asthma, cancer, schizophrenia, diabetes, high blood pressure, atherosclerosis, infection and the like.

For example, the gene of the invention can be an oncogene, whose expression within a cell induces that cell to become converted from a normal cell into a tumor cell. Further examples of genes of the invention include, but are not limited to, cytokine genes, prion genes, genes encoding molecules that induce angiogenesis, genes encoding adhesion molecules, genes encoding cell surface receptors, genes encoding proteins that are involved in metastasizing and/or invasive processes, genes of proteases as well as of molecules that regulate apoptosis and the cell cycle.

As used herein, a “biomarker” is a molecule, which corresponds to a species of a gene transcript that has a quantitatively differential concentration or level in peripheral blood cells of a subject having a disease compared to a subject not having said disease. As such, a biomarker includes a synthetic nucleic acid including cRNA, cDNA and the like. A species of a gene transcript includes any gene transcript, which is transcribed from any part of the subject's chromosomal and extra-chromosomal genome. A species of a gene transcript can be an RNA. A species of a gene transcript can be an mRNA, a cDNA or a portion thereof. Thus, a biomarker according to the present invention is a molecule that corresponds to a species of a gene transcript, which is present at an increased level or a decreased level in peripheral blood cells of at least one subject having a disease, wherein the at least one subject being a member of a closed population, when compared to the level of said transcript in peripheral blood cells of at least one subject not having said disease, wherein the subject not having the disease being a member of said closed population.

According to the present invention, the level of a gene transcript can be determined by measuring the level of the gene transcript, e.g., RNA, using semi-quantitative methods such as microarray hybridization or more quantitative methods such as quantitative RT-PCR.

When determining whether a first level of a gene transcript in a sample of peripheral blood cells of a subject having a disease is different from a second level of the gene transcript in a sample of peripheral blood cells of a subject not having the disease, a ratio between the first and second levels of the gene transcripts has to be greater or lower than 1.0. For example, a ratio of greater than 1.2, 1.5, 2, 4, 10, or 20, or lower than 0.8, 0.6, 0.2 or 0.1 indicates differential expression of the gene.

A “plurality” refers to a group of at least one or more members, more preferably to a group of at least about 10 members, and more preferably to a group of at least about 20 members.

As defined herein, the profile of a plurality of gene transcripts, which reflect gene expression levels in a particular sample is defined as a “gene transcript profile”. Comparison between gene transcript profiles of different blood cell samples can be used to discern differences in transcriptional activities. Thus, a gene transcript profile obtained from peripheral blood cells can show differences occurring between normal and diseased subjects or between untreated and treated subjects.

A gene transcript profile of at least one subject having a disease, the subject being a member of a founder population, is defined as a “reference gene transcript profile”. The reference gene transcript profile reflects the level of a plurality of gene transcripts corresponding to biomarkers of said disease. Preferably, at least two subjects are used to obtain a reference gene transcript profile. More preferably, at least 10 subjects are used to obtain a reference gene transcript profile. Most preferably, at least 25 subjects are used to obtain a reference gene transcript profile. Accordingly, a mean of the level of each one of the gene transcripts can be determined and used as the reference gene transcript profile. Alternatively and/or additionally, a range of the levels of each one of the gene transcripts corresponding to a biomarker can be determined and used as the reference gene transcript profile. It is to be understood that the reference gene transcript profile is obtained under conditions where internal and external controls are included (herein below). Additionally or alternatively, the reference gene transcript profile can be a gene transcript profile of at least one subject not having a disease, the subject not having the disease being a member of a closed population.

According to other embodiments, a gene is differentially expressed if the ratio of the mean or median level of a gene transcript in a first population as compared with the mean or median level of the gene transcript of the second population is greater or lower than 1.0.

Construction of a Microarray

A nucleic acid microarray (oligonucleotides, RNA, DNA, cDNA, PCR products or expression sequence tags) can be constructed by any method known in the art (see, for example, US Patent Application No. 2005/0042630; U.S. Pat. No. 6,607,879 which are incorporated by reference as if fully set forth herein). A nucleic acid microarray can be constructed as follows:

Nucleic acids (RNA, DNA, cDNA, PCR products or ESTs) (about 40 μl) are precipitated with 4 μl of 3M sodium acetate (pH 5.2) and 100 μl (2.5 volumes) of ethanol and stored overnight at −20° C. They are then centrifuged at 3,300 rpm at 4° C. for 1 hour. The obtained pellets are washed with 50 μl ice-cold 70% ethanol and centrifuged again for 30 minutes. The pellets are then air-dried and resuspended well in 50% dimethylsulfoxide (DMSO) or 20 μl 3×SSC overnight. The samples are then deposited either singly or in duplicate onto Gamma Amino Propyl Silane (Corning CMT-GAPS or CMT-GAP2, Catalog No. 40003, 40004) or polylysine-coated slides (Sigma Cat. No. P0425) using a robotic GMS 417 or 427 arrayer (Affymetrix, California). The boundaries of the DNA spots on the microarray are marked with a diamond scriber. The invention provides for arrays where 10-20,000 different DNAs are spotted onto a solid support to prepare an array, and also can include duplicate, triplicate or multiple DNAs.

The arrays are rehydrated by suspending the slides over a dish of warm particle free ddH₂O for approximately one minute and snap-dried on a 70-80° C. inverted heating block for 3 seconds. DNA is then UV cross-linked to the slide (Stratagene, Stratalinker) or baked at 80° C. for two to four hours. The arrays are placed in a slide rack. An empty slide chamber is prepared and filled with the following solution: 3.0 grams of succinic anhydride (Aldrich) is dissolved in 189 ml of 1-methyl-2-pyrrolidinone; immediately after the last flake of succinic anhydride dissolved, 21.0 ml of 0.2 M sodium borate is mixed in and the solution is poured into the slide chamber. The slide rack is plunged rapidly and evenly in the slide chamber and vigorously shaken up and down for a few seconds, making sure the slides never leave the solution, and then mixed on an orbital shaker for 15-20 minutes. The slide rack is then gently plunged in 95° C. ddH₂O for 2 minutes, followed by plunging five times in 95% ethanol. The slides are then air dried by allowing excess ethanol to drip onto paper towels. The arrays are then stored in the slide box at room temperature until use. Other methods for construction of microarrays as known in the art can be used.

Nucleic Acid Microarrays

Any combination of the nucleic acid sequences generated from polynucleotides complementary to regions of DNA expressed in blood are used for the construction of a microarray. A microarray according to the invention preferably comprises between 10, 100, 500, 1000, 5000, 10,000, 15,000 and 20,000 nucleic acid members. The nucleic acid members are known or novel nucleic acid sequences or any combination thereof. A microarray according to the invention is used to assay for differential gene expression profiles of genes in blood cell samples from healthy patients as compared to patients with a disease.

There are two types of controls used on microarrays. First, positive controls are genes whose expression level is invariant in disease or healthy subjects and are used to monitor target DNA binding to the slide, quality of the spotting and binding processes of the target DNA onto the slide, quality of the RNA samples, and efficiency of the reverse transcription and fluorescent labeling of the samples. Second, negative controls are external controls derived from an organism unrelated to and therefore unlikely to cross-hybridize with the sample of interest. These are used to monitor for variation in background fluorescence on the slide, and non-specific hybridization.

Preparation of Fluorescent DNA Probe from mRNA

Methods for target nucleic acid preparation are well known in the art. The cDNA can be prepared as follows:

2 μg Oligo-dT primers are annealed to 2 μg of mRNA isolated from a blood sample of a patient in a total volume of 15 μg, by heating to 70° C. for 10 min, and cooled on ice. The mRNA is reverse transcribed by incubating the sample at 42° C. for 1.5-2 hours in a 100 μl volume containing a final concentration of 50 mM Tris-HCl (pH 8.3), 75 mM KCl, 3 mM MgCl₂, 25 mM DTT, 25 mM unlabelled dNTPs, 400 units of Superscript II (200 U/μL, Gibco BRL), and 15 mM of Cy3 or Cy5 (Amersham). RNA is then degraded by addition of 15 μl of 0.1N NaOH, and incubation at 70° C. for 10 min. The reaction mixture is neutralized by addition of 15 μl of 0.1N HCl, and the volume is brought to 500 μl with TE (10 mM Tris, 1 mM EDTA), and 20 μg of Cot1 human DNA (Gibco-BRL) is added.

The labeled target nucleic acid sample is purified by centrifugation in a Centricon-30 micro-concentrator (Amicon). If two different target nucleic acid samples (e.g., two samples derived from a healthy patient vs. patient with a disease) are being analyzed and compared by hybridization to the same array, each target nucleic acid sample is labeled with a different fluorescent label (e.g., Cy3 and Cy5) and separately concentrated. For final target nucleic acid preparation 2.1 μl 20×SSC (1.5M NaCl, 150 mM NaCitrate (pH8.0)) and 0.35 μl 10% SDS is added. Other methods for probing as known in the art can be used. For example, chemiluminiscence can be used (e.g., chips of Metrigenix Ltd., gold chips, biosensors and the like.

Hybridization

Labeled nucleic acid is denatured by heating for 2 min at 100° C., and incubated at 37° C. for 20-30 min before being placed on a nucleic acid array under a 22 mm×22 mm glass cover slip. Hybridization is carried out at 65° C. for 14 to 18 hours in a custom slide chamber with humidity maintained by a small reservoir of 3×SSC. The array is washed by submersion and agitation for 2-5 min in 2×SSC with 0.1% SDS, followed by 1×SSC, and 0.1×SSC. Finally, the array is dried by centrifugation for 2 min in a slide rack in a Beckman GS-6 tabletop centrifuge in Microplus carriers at 650 rpm for 2 min. Other methods for hybridization as known in the art can be used.

Signal Detection and Data Generation

Following hybridization of an array with one or more labeled target nucleic acid samples, arrays are scanned using ScanArray Express H scanner (Perkin Elmer) and the data is acquired using the GenePix software connected to the scanner. Alternatively, other scanners and other softwares may be used.

If one target nucleic acid sample is analyzed, the sample is labeled with one fluorescent dye (e.g., Cy3 or Cy5).

After hybridization to a microarray as described herein, fluorescence intensities at the associated nucleic acid members on the microarray are determined from images taken with a scanner equipped with laser excitation sources and interference filters appropriate for the Cy3 or Cy5 fluorescence and with an appropriate program.

The presence of Cy3 or Cy5 fluorescent dye on the microarray indicates hybridization of a target nucleic acid and a specific nucleic acid member on the microarray. The intensity of Cy3 or Cy5 fluorescence represents the amount of target nucleic acid, which is hybridized to the nucleic acid member on the microarray, and is indicative of the expression level of the specific gene in the target sample.

If a nucleic acid member on the array shows a single color, it indicates that a gene corresponding to said nucleic acid member is expressed only in that blood cell sample. The appearance of both colors indicates that the gene is expressed in both blood cell samples, e.g., blood cell sample obtained from a subject having a disease and blood cell sample obtained from a subject not having the disease. The ratios of Cy3 and Cy5 fluorescence intensities, after normalization, are indicative of differences of expression levels of the associated nucleic acid members in the two samples for comparison. A ratio of expression not equal to 1.0 is used as an indication of differential gene expression.

Identification of genes differentially expressed in blood cell samples from patients with disease as compared to healthy patients or as compared to patients without said disease is determined by statistical analysis of the gene expression profiles from healthy patients or patients without disease compared to patients with disease using the “R” language, the “Bioconductor” software project for analysis and comprehension of genomic data (see, for example, Gentleman, R. C. et al., Genome Biol. 5(10):R80, 2004. Epub 2004 Sep. 15; and the Wilcox Mann Whitney rank sum test). Other statistical tests can also be used (see, for example, Sokal and Rohlf (1987) Introduction to Biostatistics 2nd edition, WH Freeman, New York, which is incorporated herein in their entirety).

In order to facilitate ready access, e.g. for comparison, review, recovery and/or modification, the expression profiles of patients with disease and/or patients without disease or healthy patients can be recorded in a database.

As would be understood by a person skilled in the art, comparison as between the expression profile of a test patient with expression profiles of patients with a disease, expression profiles of patients with a certain stage or degree of progression of said disease, without said disease, or healthy individuals so as to diagnose or prognose said test patient can occur via expression profiles generated concurrently or non concurrently. It would be understood that expression profiles can be stored in a database to allow said comparison.

Use of Expression Profiles for Diagnostic Purposes

As would be understood to a person skilled in the art, one can utilize sets of genes, which have been identified as differentially expressed in a disease as described above in order to characterize an unknown sample as having said disease or not having said disease.

The diagnosing or prognosing may thus be performed by comparing the expression level of one or more genes, three or more genes, five or more genes, ten or more genes, twenty or more genes, fifty or more genes, one hundred or more genes, two hundred or more genes, or all of the genes disclosed for the specific disease in question.

Data Acquisition and Analysis of Differentially Expressed EST Sequences

The differentially expressed EST sequences are then searched against available databases, including the “Reference Sequence” (RefSeq) collection and the “UniGene” system for automatically partitioning “GenBank” sequences into a non-redundant set of gene-oriented clusters, “nt”, “nr”, “est”, “gss” and “htg” databases available through NCBI to determine putative identities for ESTs matching to known genes or other ESTs. Functional characterization of ESTs with known gene matches is made according to any known method. For example, differentially expressed EST sequences are compared to the non-redundant Genbank/EMBL/DDBJ and dbEST databases using the BLAST algorithm (Altschul S. F., et al., J. Mol. Biol. 215: 403-410, 1990).

Genes are identified from ESTs according to known methods. To identify novel genes from an EST sequence, the EST should preferably be at least 100 nucleotides in length, and more preferably 150 nucleotides in length, for annotation. Preferably, the EST exhibits open reading -frame characteristics (i.e., can encode a putative polypeptide).

Because of the completion of the Human Genome Project, a specific EST, which matches with a genomic sequence can be mapped onto a specific chromosome based on the chromosomal location of the genomic sequence. However, no function may be known for the protein encoded by the sequence and the EST would then be considered “novel” in a functional sense. In one aspect, the invention is used to identify a novel differentially expressed EST, which is part of a larger known sequence for which no function is known. Alternatively, or additionally, the EST can be used to identify an mRNA or polypeptide encoded by the larger sequence as a diagnostic or prognostic biomarker of a disease.

Identified genes can be catalogued according to their putative function. Functional characterization of ESTs with known gene matches is preferably made according to the categories described by Hwang et al. (Circulation 96: 4146-4203, 1997).

Known Nucleic Acid Sequences or ESTs and Novel Nucleic Acid Sequences or ESTs

An EST that exhibits a significant match (>90% identity in at least 200 bp long) to at least one existing sequence in an existing nucleic acid sequence database is characterized as a “known” sequence according to the invention. Within this category, some known ESTs match to existing sequences, which encode polypeptides with known function(s) and are referred to as a “known sequence with a function”. Other “known” ESTs exhibit a significant match to existing sequences, which encode polypeptides of unknown function(s) and are referred to as a “known sequence with no known function”.

EST sequences, which have no significant match (less than 65% identity) to any existing sequence in the above cited available databases are categorized as novel ESTs. To identify a novel gene from an EST sequence, the EST is preferably at least 150 nucleotides in length. More preferably, the EST encodes at least part of an open reading frame, that is, a nucleic acid sequence between a translation initiation codon and a termination codon, which is potentially translated into a polypeptide sequence.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLE 1

### Determination of Gene Expression Levels in White Blood Cells

Selecting Healthy Subjects and Subjects Having Asthma

The Cochin Jews are a closed population that lived isolated for many generations in Cochin, a small city within the Malabar region in India. The intermarriages with the local population as well as with other Indian Jews were scarce and most of the marriages were arranged within the community. The Cochin Jews immigrated to Israel in the late 1950's and settled in two main geographical areas, in some villages close to Jerusalem and in the Negev Desert.

Subjects were diagnosed as having asthma phenotypes by performing the following tests: allergy skin tests, blood tests for IgE and eosinophils levels, pulmonary function tests, response to asthma drugs such as ventoline and methacholine challenge test according to the American Thoracic Society Standards (Amer. J. Respir. Crit. Care Med. 152: 1107-1136, 1995).

The Cochin population has been shown to have higher incidence of asthma and allergy (see Table 1) as compared to the incidence of these conditions in the non-Cochin population.

TABLE 1
Prevalence of asthma and allergy in the Cochin and
non-Cochin populations.

| | Total | Cochins- | Cochins- | Non-Cochins- |
|---|---|---|---|---|
| | Cochins | desert | Jerusalem | Jerusalem |
| N | 834 | 353 | 481 | 149 |
| Mean age, | 32.7 ± 20.9 | | | |
| years | | | | |
| Asthma total | 198 (23.7%) | 66 (18.7%) | 132 (27.4%) | 13 (8.7%) |
| Allergic | 117 (14%) | 27 (7.6%) | 90 (18.7%) | 7 (4.7%) |
| asthma | | | | |
| Non-allergic | 81 (9.7%) | 39 (11.0%) | 42 (8.7%) | 6 (4%) |
| asthma | | | | |
| Allergy total | 246 (29.5%) | 80 (22.7%) | 166 (34.5%) | 10 (7%) |
| Allergy with | 129 (15.5%) | 53 (15%) | 76 (15.8%) | 3 (2%) |
| no asthma | | | | |
| Allergy and/ | 327 (39.2%) | 119 (33.7%) | 208 (43.2%) | 16 (10.7%) |
| or asthma | | | | |

As shown in Table 1, among the Cochin Jewish population, the prevalence of asthma was 23.7% and of allergy 29.5%. The prevalence of asthma and allergy in the non-Cochin population was 8.7% and 7%, respectively. There was no difference in the prevalence of asthma between males (25.3%) and females (22.2%). In addition, the prevalence of allergic asthma was significantly higher than non-allergic asthma (14% vs. 9.7%; p>0.001). In contrast, in the non-Cochin population, the prevalence of allergic asthma and non-allergic asthma was very similar (4.7% vs. 4%).

The following groups were used for the experiment:
2. Cochin subjects (both parents are Cochins) having from asthma—19 subjects;
3. Cochin subjects (both parents are Cochins) with no evidence of asthma—19 subjects;
4. Non-Cochin Jews having asthma—20 subjects; and
5. Non-Cochin Jews with no evidence of asthma—25 subjects.

Most of the subjects of the Cochin population were chosen in a way that healthy and asthma subjects were originate from the same family (e.g., parents, children and siblings) in order to further decrease the variations between individuals and hence to increase the statistical significance of the study.

Leukocyte Purification

Venous blood (50 ml) was collected from each individual into tubes (Benkton Dickenson Vacutainer), which contained K-EDTA as an anticoagulant, and placed immediately on ice. Two ml of blood were removed and used for differential cell counting and IgE determination.

Leukocyte purification was performed within 5 minutes after blood withdrawal as follows:

15 ml Ficoll-400 (Amersham) were placed in 50 ml plastic tubes (Nunc; 4 tubes per volunteer). Fifty ml of the freshly collected blood (5 min after drawing) were diluted with phosphate buffered saline (without Ca++ and Mg++; Sigma) to a final volume of 100 ml and carefully placed over the Ficoll column. The columns were centrifuged in a cooled eppendorff centrifuge (model 5810, rotor A-4-81) for 20 min at 1000×g. Immediately thereafter, four ml of serum of each sample were separated and frozen in liquid nitrogen.

The buffy coat containing the leukocytes was separated by centrifugation at 100×g in a cooled eppendorff centrifuge (model 5810, rotor A-4-81) and washed once with 2 ml phosphate buffered saline (without Ca++ and Mg++; Sigma). The purified leukocytes (between 6 to 12 ml) were placed in 4 ml cryotubes (Nunc) containing 2 ml of ice cold Trizol (Invitrogen), vortexed and frozen in liquid nitrogen. The leukocytes were kept frozen until RNA purification.

RNA Purification

Total RNA of leukocytes was purified using Trizol (Invitrogene) and Phase Lock Gel (Invitrogen) according to the manufacturer's procedure. This method achieved high yield of RNA. Briefly, the leukocytes were thawed at room temperature. The ratio of cell/trizol was 5×10⁶ cells, 1 ml of Trizol.

Phase lock gel was added to the cell lisate (0.2 ml of phase lock gel per 1 ml of Trizol) and incubated for 15 min at room temperature. The tubes were centrifuged at 3250×g in a cooled eppendorff centrifuge (model 5810, rotor A-4-81) for 15 min at 4° C. The pure aqueous phase was transferred to a new tube and 0.5 ml of isopropyl alcohol were added per 1 ml of Trizol. The samples were mixed by vortexing, incubated for 15 min at room temperature, and centrifuged at 3250×g in a cooled eppendorff centrifuge (model 5810, rotor A-4-81) for 45 min at 4° C. to precipitate the RNA. The supernatant was discarded and the pellet was washed with 30 ml of ice cold 75% ethanol/water. The suspension was centrifuged for 15 min at 3250×g in a cooled eppendorff centrifuge (model 5810, rotor A4-81) at 4° C., the ethanol was decanted and the pellet was resuspended again in ice cold 75% ethanol/water and centrifuged for additional 5 min. The residual ethanol was removed and the RNA was immediately resuspended in 800 μl of nuclease free water and transferred to a 1.5 ml eppendorff tube.

For RNA purification, 400 μl of 7.5 M lithium chloride were added to each tube, mixed and incubated at −20° C. overnight.

Purification of RNA bv LiCl Precipitation

The samples were thawed, centrifuged at 15000 rpm for 15 min at 4° C. (eppendorff microcentrifuge), and the liquid was removed. The samples were washed twice with 1 ml 75% ethanol and ethanol traces were removed. The RNA was resuspended in 250 μl of nuclease free water and its concentration was brought to 5.5 mg/ml either by further dilution or by sodium acetate precipitation (see Bowtell, D. and Sambrook, J. Eds. Cold Spring Harbor Laboratory Press)

All samples had a 260/280 ratio of at least 1.8 and appear non-degraded when electropboresed on 1% agarose gel. The samples (9 μl aliquots) were kept at −80° C. until use.

RNA Amplification

Five μg of RNA from each of the samples were amplified using the Message-AmpII kit (Ambion, USA).

Fluorescence Labeling of the Samples for Microarray

All samples were fluorescently labeled using the fluorescent dyes Cy3 and Cy5 as follows: 50 μg of each RNA sample were combined with 1 μl of random primers (Invitrogene USA) in a total volume of 10 μl and incubated at 70° C. for 10 minutes for annealing, after which centrifugation was performed.

Two separate reaction mixtures were prepared for each sample. Each reaction was performed according with Superscriptll kit instructions. Briefly, a mixture containing 4 μl of 10× reaction buffer (Invitrogene, USA), 2 μl of 0.1M DTT (Invitrogene, USA), 2 μl of 10× low cytosine dNTP (Invitrogene, USA), 2 μl of Cy3 or Cy5 dCTP (Perkin Elmer, USA), 0.5 μl RNAse inhibitor (Invitrogene, USA), and 1 μl SuperScriptII reverse transcriptase (Invitrogene, USA). Each reaction mixture was added to an RNA sample+random primers (Invitrogene, USA), mixed and placed in a 42° C. water bath for 45 minutes followed by a brief centrifugation to collect condensation in the tubes. To each tube an additional 1 μl of SuperScriptII reverse transcriptase (Invitrogene USA) was added and the samples were further incubated at 42° C. for 45 minutes. Thereafter, the tubes were placed at 95° C. for two minutes and then place on ice.

cDNA Purification

The RNA was degraded using RNAse I (Promega, USA) and the cDNA was purified using QlAquick PCR purification kit (Quiagen) according to the manufacturer's procedure. The purified cDNA samples were concentrated on Microcon YM50 columns (Millipore) by centrifuging the samples applied to the columns for 2 min at 12400 rpm (eppendorff microfuge) at room temperature, inverting the columns, and then centrifuging again at 3000×g in an eppendorff microfuge to collect the cDNA. The volume of each sample was brought to 15 μl.

Hybridization to Microarrays

A library of about 41500 cDNA clones derived from the I.M.A.G.E consortium was purchased from Research Genetics (40K Human set, Research Genomics, USA) by the Van Andel Research Institute and sequence verified by Research Genomics (USA). The library was printed in two microarray slides denominated Human Array A and Human Array B. The two arrays together comprised the whole transcriptosome. Each of the cDNA fragments printed in the array were in average 1000 bp (ranging from 500 to 2500 bp). The library was printed using a custom built instrument built by Beta Integrated Concepts, Byron Center, MI, according with the provider's manual using Point Technologies (USA) PT-3000 split pins.

Each sample was hybridized to both Human Array A and Human Array B. To each array two different targets were hybridized, the sample in study and a common control sample labeled with either Cy3 or Cy5 dye.

The common control sample was created by the amplification of the same library used to print the array (40K Human set, Research Genomics, USA) and was labeled as described for the experimental samples. The use of a common control allows for the use of one control system for all the arrays in the study.

The microarray hybridization was performed according to well-known procedures (see, for example, Bowtell, D. and Sambrook, J. Eds. Cold Spring Harbor Laboratory Press.)

Data Acquisition and Analysis

The arrays were scanned in an Agilent Microarray Scanner, model G2565BA (Agilent, USA, within 5-6 hours after hybridization, the data acquisition, and Image analysis was performed in GenePix version 5.1.0.1 software connected to the scanner.

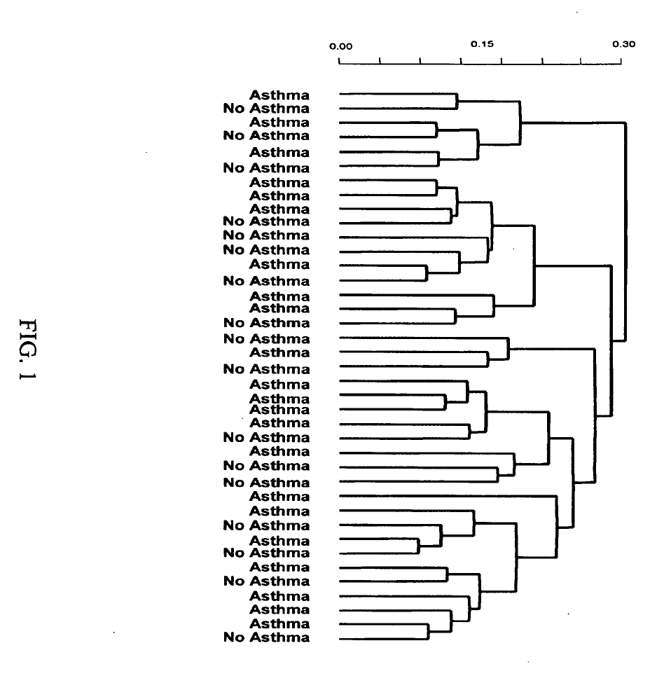

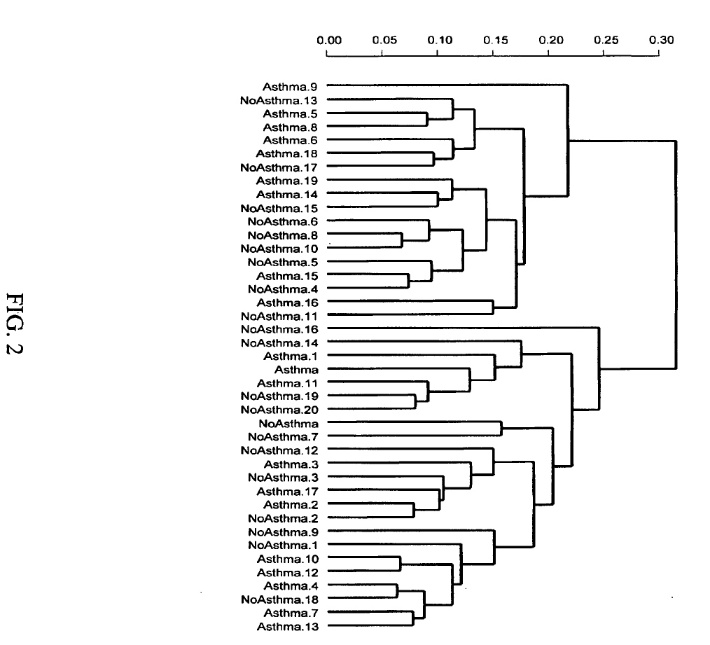

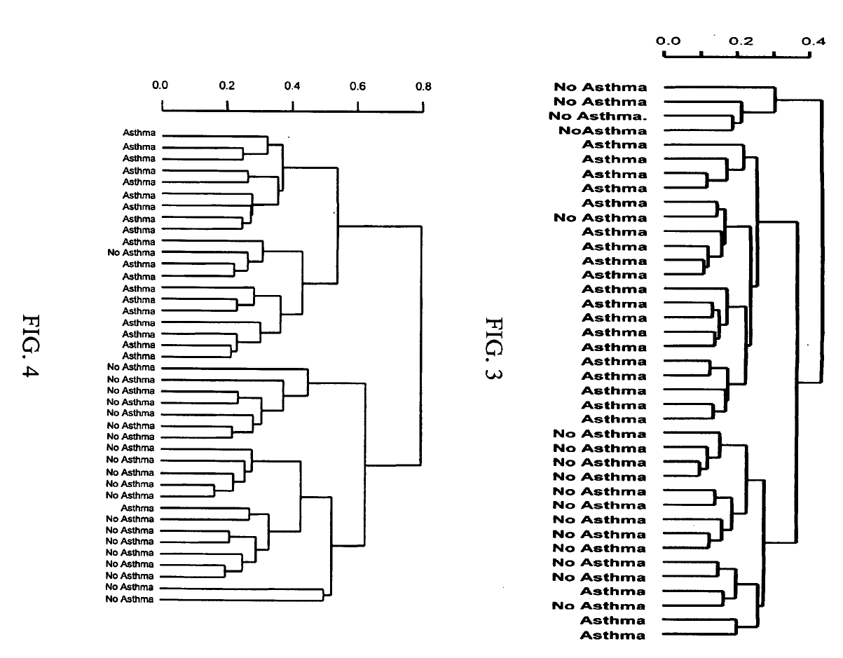

Following acquisition, quality control and statistical analysis of microarray data were done using the “R” language and environment version 2.01 (Ihaka R and Gentleman R, J. Comput. Graph. Stat. 5(3): 299-314, 1996) and the “Bioconductor” software project for the analysis and comprehension of genomic data (Gentleman, R. C. et al., Genome Biol. 5(10): R80, 2004. Epub 2004 Sep. 15). The analysis protocol consisted of clustering all the genes regulated in arrays A and B in the closed population as shown in FIGS. 1 and 2. It is clearly seen in these figures that the majority of the genes are similar and thus there is no clear separation between asthma and non-asthma individuals. The second step was extracting or eliminating the genes that are common to the afflicted and non afflicted individuals in the closed population, followed by clustering of the remaining genes in the arrays as shown in FIGS. 3 and 4. Following the extraction of the common genes it is clearly seen that the separation between asthma and non-asthma is clear. The statistic significance of each gene transcript between asthma and non-asthma individuals was calculated and expressed as P values for each gene in Tables 2, 3 and 4. Statistically significant genes were those that showed P values of 0.05 and below.

The gene transcripts, which were specifically regulated in the Cochin closed population suffering from asthma and which were not regulated in the asthma-free Cochin population, were defined as “asthma primary gene expression profile”.

The asthma primary gene expression profile was then compared to the gene expression profile of asthma individuals in the open population and only gene transcripts that were regulated in the closed population and open population were selected and were defined as “final asthma gene expression profile”.

Table 2 lists the genes that were identified as biomarkers for asthma.

TABLE 2
List of known mRNAs differentially expressed in asthma subjects

| SEQ ID NO | Regulation | Accession on Chip | Refseq_gi | Refseq_Accession | Refseq_Description |
|---|---|---|---|---|---|
| 1 | UP | R43320 | 10567815 | NM_020988 | Homo sapiens guanine nucleotide binding protein (G protein), alpha activating activity |
| | | | | | polypeptide O (GNAO1), transcript variant 1, mRNA |
| 2 | DOWN | AA035450 | 10835022 | NM_002222 | Homo sapiens inositol 1,4,5-triphosphate receptor, type 1 (ITPR1), mRNA |
| 3 | UP | AA702335 | 10864012 | NM_021200.1 | Homo sapiens pleckstrin homology domain containing, family B (evectins) member 1 |
| | | | | | (PLEKHB1), mRNA |
| 4 | UP | AA398356 | 11034818 | NM_020645 | Homo sapiens nuclear receptor interacting protein 3 (NRIP3), mRNA |
| 5 | DOWN | AA464417 | 11995467 | NM_021034.1 | Homo sapiens interferon induced transmembrane protein 3 (1-8 U) (IFITM3), mRNA |
| 6 | DOWN | AI187357 | 12232456 | NM_022774.1 | Homo sapiens hypothetical protein FLJ21144 (FLJ21144), mRNA |
| 7 | DOWN | AA922832 | 12545399 | NM_002162.2 | Homo sapiens intercellular adhesion molecule 3 (ICAM3), mRNA |
| 8 | UP | AA447588 | 13514808 | NM_004660 | Homo sapiens DEAD (Asp-Glu-Ala-Asp) box polypeptide 3, Y-linked (DDX3Y), mRNA |
| 9 | DOWN | AA703557 | 13540489 | NM_021813.1 | Homo sapiens BTB and CNC homology 1, basic leucine zipper transcription factor 2 (BACH2), |
| | | | | | mRNA |
| 10 | DOWN | AA262351 | 13775237 | NM_031310 | Homo sapiens plasmalemma vesicle associated protein (PLVAP), mRNA |
| 11 | DOWN | AA989257 | 13994241 | NM_031892.1 | Homo sapiens SH3-domain kinase binding protein 1 (SH3KBP1), mRNA |
| 12 | UP | AA629117 | 14042973 | NM_032041.1 | Homo sapiens neurocalcin delta (NCALD), mRNA |
| 13 | DOWN | H15274 | 14149689 | NM_015453 | Homo sapiens THUMP domain containing 3 (THUMPD3), mRNA |
| 14 | UP | AA459402 | 14211539 | NM_020963 | Homo sapiens Mov10, Moloney leukemia virus 10, homolog (mouse) (MOV10), mRNA |
| 15 | DOWN | AA426092 | 14249371 | NM_032744 | Homo sapiens chromosome 6 open reading frame 105 (C6orf105), mRNA |
| 16 | UP | H89490 | 14249505 | NM_032814 | Homo sapiens hypothetical protein FLJ14627 (FLJ14627), mRNA |
| 17 | UP | AI017101 | 14790189 | NM_015001.2 | Homo sapiens spen homolog, transcriptional regulator (Drosophila) (SPEN), mRNA |
| 18 | DOWN | R56211 | 15451788 | NM_002609 | Homo sapiens platelet-derived growth factor receptor, beta polypeptide (PDGFRB), mRNA |
| 19 | DOWN | AA453578 | 15529971 | NM_033412 | Homo sapiens mitochondrial carrier triple repeat 1 (MCART1), mRNA |
| 20 | UP | AA458932 | 15529981 | NM_033416 | Homo sapiens IMP4, U3 small nucleolar ribonucleoprotein, homolog (yeast) (IMP4), mRNA |
| 21 | UP | AA278384 | 16306490 | NM_001786.2 | Homo sapiens cell division cycle 2, G1 to S and G2 to M (CDC2), transcript variant 1, mRNA |
| 22 | DOWN | AA677076 | 16579891 | NM_052834 | Homo sapiens WD repeat domain 7 (WDR7), transcript variant 2, mRNA |
| 23 | DOWN | AA625981 | 17149837 | NM_000801 | Homo sapiens FK506 binding protein 1A, 12 kDa (FKBP1A), transcript variant 12B, mRNA |
| 24 | UP | AA453616 | 17572806 | NM_031916 | Homo sapiens ropporin 1-like (ROPN1L), mRNA |
| 25 | DOWN | AA620890 | 17864091 | NM_018897 | Homo sapiens dynein, axonemal, heavy polypeptide 7 (DNAH7), mRNA |
| 26 | UP | AA450001 | 17921983 | NM_004375.2 | Homo sapiens COX11 homolog, cytochrome c oxidase assembly protein (yeast) (COX11), |
| | | | | | mitochondrial protein, mRNA |
| 27 | DOWN | AI369867 | 17986269 | NM_016574.2 | Homo sapiens dopamine receptor D2 (DRD2), transcript variant 2, mRNA |
| 28 | DOWN | AA884902 | 17999529 | NM_005205.2 | Homo sapiens cytochrome c oxidase subunit VIa polypeptide 2 (COX6A2), nuclear gene nuclear gene encoding |
| | | | | | encoding mitochondrial protein, mRNA |
| 29 | UP | AI001846 | 18378734 | NM_014810.2 | Homo sapiens centrosome-associated protein 350 (CAP350), mRNA |
| 30 | DOWN | H09076 | 18491007 | NM_000775 | Homo sapiens cytochrome P450, family 2, subfamily J, polypeptide 2 (CYP2J2), mRNA |
| 31 | DOWN | AA430052 | 18497285 | NM_020865 | Homo sapiens DEAH (Asp-Glu-Ala-His) box polypeptide 36 (DHX36), mRNA |
| 32 | DOWN | AA490612 | 18553076 | XM_087200.1 | PREDICTED: Homo sapiens hypothetical LOC151443 (LOC151443), mRNA |
| 33 | DOWN | AA663884 | 18765734 | NM_130811 | Homo sapiens synaptosomal-associated protein, 25 kDa (SNAP25), transcript variant 2, mRNA |
| 34 | UP | AA131162 | 19718758 | NM_133337.1 | Homo sapiens fer-1-like 3, myoferlin (C. elegans) (FER1L3), transcript variant 2, mRNA |
| 35 | DOWN | N27023 | 19747268 | NM_130776 | Homo sapiens X antigen family, member 3 (XAGE3), transcript variant 2, mRNA |
| 36 | UP | AA449234 | 19913395 | NM_134269 | Homo sapiens smoothelin (SMTN), transcript variant 2, mRNA |
| 37 | DOWN | AA460719 | 19923448 | NM_016025 | Homo sapiens DORA reverse strand protein 1 (DREV1), mRNA |
| 38 | DOWN | AA122022 | 19923496 | NM_019063 | Homo sapiens echinoderm microtubule associated protein like 4 (EML4), mRNA |
| 39 | UP | N51752 | 19923854 | NM_032796 | Homo sapiens synapse associated protein 1, SAP47 homolog (Drosophila) (SYAP1), mRNA |
| 40 | UP | AA428196 | 20127494 | NM_006237 | Homo sapiens POU domain, class 4, transcription factor 1 (POU4F1), mRNA |
| 41 | UP | AA424948 | 20336296 | NM_022779 | Homo sapiens DEAD (Asp-Glu-Ala-Asp) box polypeptide 31 (DDX31), transcript variant 1, |
| | | | | | mRNA |
| 42 | UP | AA291513 | 20336472 | NM_001707 | Homo sapiens B-cell CLL/lymphoma 7B (BCL7B), transcript variant 1, mRNA |
| 43 | DOWN | AA402766 | 20357549 | NM_014313 | Homo sapiens small membrane protein 1 (SMP1), mRNA |
| 44 | DOWN | AA487243 | 21071076 | NM_014992 | Homo sapiens dishevelled associated activator of morphogenesis 1 (DAAM1), mRNA |
| 45 | DOWN | AA598780 | 21237785 | NM_014776 | Homo sapiens G protein-coupled receptor kinase interactor 2 (GIT2), transcript variant 3, mRNA |
| 46 | DOWN | W32408 | 21265083 | NM_020236 | Homo sapiens mitochondrial ribosomal protein L1 (MRPL1), nuclear gene encoding mitochondrial protein, mRNA |
| 47 | DOWN | AA971699 | 21327694 | NM_139264.1 | Homo sapiens ADAMTS-like 1 (ADAMTSL1), transcript variant 3, mRNA |
| 48 | UP | AA666316 | 21361102 | NM_003705.2 | Homo sapiens solute carrier family 25 (mitochondrial carrier, Aralar), member 12 (SLC25A12), |
| | | | | | mRNA |
| 49 | DOWN | H03040 | 21361391 | NM_006905 | Homo sapiens pregnancy specific beta-1-glycoprotein 1 (PSG1), mRNA |
| 50 | DOWN | R56877 | 21361486 | NM_015595.2 | Homo sapiens Src homology 3 domain-containing guanine nucleotide exchange factor (SGEF), |
| | | | | | mRNA |
| 51 | DOWN | T70901 | 21361540 | NM_016400 | Homo sapiens Huntingtin interacting protein K (HYPK), mRNA |
| 52 | DOWN | AA446864 | 21361616 | NM_019000 | Homo sapiens hypothetical protein FLJ20152 (FLJ20152), mRNA |
| 53 | UP | AA630354 | 21361698 | NM_020126 | Homo sapiens sphingosine kinase 2 (SPHK2), mRNA |
| 54 | DOWN | T85191 | 21361813 | NM_019118 | Homo sapiens hypothetical protein RP4-622L5, (RP4-622L5), mRNA |
| 55 | DOWN | AA862473 | 21361833 | NM_020372.2 | Homo sapiens solute carrier family 22 (organic cation transporter), member 17 (SLC22A17), transcript variant 1, mRNA |
| 56 | UP | AA465381 | 21361958 | NM_025181.2 | Homo sapiens solute carrier family 35, member F5 (SLC35F5), mRNA |
| 57 | UP | W84815 | 21362109 | NM_022445 | Homo sapiens thiamin pyrophosphokinase 1 (TPK1), mRNA |
| 58 | DOWN | N20072 | 21389336 | NM_144563 | Homo sapiens ribose 5-phosphate isomerase A (ribose 5-phosphate epimerase) (RPIA), mRNA |
| 59 | DOWN | AA913839 | 21396501 | NM_022142.3 | Homo sapiens epididymal sperm binding protein 1 (ELSPBP1), mRNA |
| 60 | UP | AI681015 | 21450784 | NM_004232.2 | Homo sapiens suppressor of cytokine signaling 6 (SOCS6), mRNA |
| 61 | UP | AI025520 | 21493038 | NM_139289.1 | Homo sapiens A kinase (PRKA) anchor protein 4 (AKAP4), transcript variant 2, mRNA |
| 62 | UP | H29322 | 21536281 | NM_003656 | Homo sapiens calcium/calmodulin-dependent Protein kinase I (CAMK1), mRNA |
| 63 | DOWN | AA286814 | 21536339 | NM_012468.3 | Homo sapiens T-cell leukemia/lymphoma 6 (TCL6), transcript variant TCL6a1, mRNA |
| 64 | UP | AA521292 | 21536375 | NM_005502.2 | Homo sapiens ATP-binding cassette, sub-family A (ABC1), member 1 (ABCA1), mRNA |
| 65 | UP | AI281149 | 21536483 | NM_005339.3 | Homo sapiens huntingtin interacting protein 2 (HIP2), mRNA |
| 66 | DOWN | AA039929 | 21614500 | NM_144780 | Homo sapiens degenerative spermatocyte homolog 1, lipid desaturase (Drosophila) (DEGS1), transcript variant 2, mRNA |
| 67 | UP | AI276917 | 21699059 | NM_144976.1 | Homo sapiens zinc finger protein 564 (ZNF564), mRNA |
| 68 | UP | AI024655 | 21717806 | NM_145647.1 | Homo sapiens unknown MGC21654 product (MGC21654), mRNA |
| 69 | DOWN | AA857804 | 21735568 | NM_022111.2 | Homo sapiens claspin homolog (Xenopus laevis) (CLSPN), mRNA |
| 70 | UP | AA677317 | 22091459 | NM_014248.2 | Homo sapiens ring-box 1 (RBX1), mRNA |
| 71 | DOWN | T50747 | 22095354 | NM_017693.2 | Homo sapiens basic, immunoglobulin-like variable motif containing (BIVM), mRNA |
| 72 | UP | AA496047 | 22538408 | NM_145893.1 | Homo sapiens ataxin 2-binding protein 1 (A2BP1), transcript variant 3, mRNA |
| 73 | UP | AI364359 | 22547196 | NM_007129.2 | Homo sapiens Zic family member 2 (odd-paired homolog, Drosophila) (ZIC2), mRNA |
| 74 | DOWN | AA453585 | 22749104 | NM_152533.1 | Homo sapiens chromosome 3 open reading frame 22 (C3orf22), mRNA |
| 75 | UP | AA457490 | 22749478 | NM_152755 | Homo sapiens hypothetical protein MGC40499 (MGC40499), mRNA |
| 76 | DOWN | T90560 | 23097284 | NM_152299 | Homo sapiens hypothetical protein 384D8_6 (384D8-2), mRNA |
| 77 | DOWN | R22420 | 23111058 | NM_022776 | Homo sapiens oxysterol binding protein-like 11 (OSBPL11), mRNA |
| 78 | UP | AA485677 | 23308730 | NM_003302 | Homo sapiens thyroid hormone receptor interactor 6 (TRIP6), mRNA |
| 79 | UP | AI473374 | 23510417 | NM_153189.1 | Homo sapiens sperm adhesion molecule 1 (PH-20 hyaluronidase. zona pellucida binding) |
| | | | | | (SPAM1), transcript variant 2, mRNA |
| 80 | DOWN | N51304 | 23821014 | NM_016544 | Homo sapiens Ras-associated protein Rap1 (RBJ), mRNA |
| 81 | DOWN | H65034 | 24429565 | NM_002603 | Homo sapiens phosphodiesterase 7A (PDE7A), transcript variant 1, mRNA |
| 82 | DOWN | AA678306 | 24431986 | NM_018452 | Homo sapiens chromosome 6 open reading frame 35 (C6orf35), mRNA |
| 83 | UP | AA676673 | 24431998 | NM_022836.2 | Homo sapiens DNA cross-link repair 1B (PSO2 homolog, S. cerevisiae) (DCLRE1B), mRNA |
| 84 | UP | AA634427 | 24475639 | NM_018405.2 | Homo sapiens hypothetical protein, clone 2746033 (HSA272196), mRNA |
| 85 | DOWN | AA454564 | 24475811 | NM_019557 | Homo sapiens hypothetical protein RP1-317E23 (LOC56181), mRNA |
| 86 | DOWN | AA916325 | 24497582 | NM_003739 | Homo sapiens aldo-keto reductase family 1, member C3 (3-alpha hydroxysteroid |
| | | | | | dehydrogenase, type II) (AKR1C3), mRNA |
| 87 | UP | AA455242 | 24497619 | NM_014230 | Homo sapiens signal recognition particle 68 kDa (SRP68), mRNA |
| 88 | DOWN | AA777219 | 25777693 | NM_171982.1 | Homo sapiens tripartite motif-containing 35 (TRIM35), transcript variant 2, mRNA |
| 89 | UP | AA610005 | 25777695 | NM_021253.2 | Homo sapiens tripartite motif-containing 39 (TRIM39), transcript variant 1, mRNA |
| 90 | DOWN | AI279844 | 25914748 | NM_033450.2 | Homo sapiens ATP-binding cassette, sub-family C (CFTR/MRP), member 10 (ABCC10), mRNA |
| 91 | DOWN | AA864875 | 26667173 | NM_172178.1 | Homo sapiens mitochondrial ribosomal protein L42 (MRPL42), nuclear gene encoding |
| | | | | | mitochondrial protein, transcript variant 3, mRNA |
| 92 | DOWN | AA983462 | 27436949 | NM_005573.2 | Homo sapiens lamin B1 (LMNB1), mRNA |
| 93 | DOWN | AA976171 | 27436956 | NM_012301.2 | Homo sapiens atrophin-1 interacting protein 1 (AIP1), mRNA |
| 94 | UP | R49756 | 27436961 | NM_003471.2 | Homo sapiens potassium voltage-gated channel, shaker-related subfamily, beta member 1 |
| | | | | | (KCNAB1), transcript variant 2, mRNA |
| 95 | UP | AI347604 | 27734778 | NM_173666.1 | Homo sapiens hypothetical protein FLJ33977 (FLJ33977), mRNA |
| 96 | DOWN | H09541 | 27734858 | NM_173662 | Homo sapiens ring finger protein 175 (RNF175), mRNA |
| 97 | DOWN | AA405558 | 27735098 | NM_173497 | Homo sapiens HECT domain containing 2 (HECTD2), transcript variant 2, mRNA |
| 98 | DOWN | AA127221 | 27894327 | NM_003856 | Homo sapiens interleukin 1 receptor-like 1 (IL1RL1), transcript variant 2, mRNA |
| 99 | DOWN | AA424734 | 28212219 | NM_175569 | Homo sapiens Xg blood group (pseudoautosomal boundary-divided on the X chromosome) |
| | | | | | (XG), mRNA |
| 100 | DOWN | AA780712 | 28372492 | NM_003588.2 | Homo sapiens cullin 4B (CUL4B), mRNA |
| 101 | DOWN | R40567 | 28558972 | NM_004229 | Homo sapiens cofactor required for Sp1 transcriptional activation, subunit 2, 150 kDa (CRSP2), |
| | | | | | mRNA |
| 102 | DOWN | H05445 | 28872807 | NM_002045 | Homo sapiens growth associated protein 43 (GAP43), mRNA |
| 103 | UP | AA706969 | 28872813 | NM_017893 | Homo sapiens sema domain, immunoglobulin domain (Ig), transmembrane domain (TM) and |
| | | | | | short cytoplasmic domain, (semaphorin) 4G (SEMA4G), mRNA |
| 104 | DOWN | AI167231 | 29171743 | NM_003712.2 | Homo sapiens phosphatidic acid phosphatase type 2C (PPAP2C), transcript variant 1, mRNA |
| 105 | DOWN | W67243 | 29244580 | NM_015336 | Homo sapiens zinc finger, DHHC domain containing 17 (ZDHHC17), mRNA |
| 106 | UP | T62529 | 29294623 | NM_177542 | Homo sapiens small nuclear ribonucleoprotein D2 polypeptide 16.5 kDa (SNRPD2), transcript |
| | | | | | variant 2, mRNA |
| 107 | UP | R00103 | 29826320 | NM_014189.2 | Homo sapiens adducin 1 (alpha) (ADD1), transcript variant 2, mRNA |
| 108 | DOWN | N51651 | 29826340 | NM_015099 | Homo sapiens calmodulin binding transcription activator 2 (CAMTA2), mRNA |
| 109 | DOWN | R38540 | 30410709 | NM_019053 | Homo sapiens SEC15-like 1 (S. cerevisiae) (SEC15L1), mRNA |
| 110 | DOWN | AI017130 | 30474868 | NM_032023.3 | Homo sapiens Ras association (RaIGDS/AF-6) domain family 4 (RASSF4), transcript variant 1, |
| | | | | | mRNA |
| 111 | DOWN | R39111 | 31317227 | NM_004430 | Homo sapiens early growth response 3 (EGR3), mRNA |
| 112 | UP | AA482251 | 31317308 | NM_012398 | Homo sapiens phosphatidylinositol-4-phosphate 5-kinase, type I, gamma (PIP5K1C), mRNA |
| 113 | UP | R47938 | 31341099 | NM_173795 | Homo sapiens hypothetical protein FLJ32096 (FLJ32096), mRNA |
| 114 | DOWN | AA903140 | 31341355 | NM_174902.2 | Homo sapiens hypothetical protein LOC143458 (LOC143458), mRNA |
| 115 | UP | AA927911 | 31341399 | NM_174939.2 | Homo sapiens hypothetical protein MGC39681 (MGC39681), mRNA |
| 116 | UP | AA187287 | 31343354 | NM_177453 | Homo sapiens progestin and adipoQ receptor family member III (PAQR3), mRNA |
| 117 | UP | H16796 | 31377664 | NM_031954 | Homo sapiens potassium channel tetramerisation domain containing 10 (KCTD10), mRNA |
| 118 | DOWN | AA521035 | 31377836 | NM_024622.2 | Homo sapiens hypothetical protein FLJ21901 (FLJ21901), mRNA |
| 119 | DOWN | AI675889 | 31542152 | NM_000905.2 | Homo sapiens neuropeptide Y (NPY), mRNA |
| 120 | UP | AA410375 | 31542848 | NM_006877 | Homo sapiens guanosine monophosphate reductase (GMPR), mRNA |
| 121 | UP | AI215689 | 31543168 | NM_032701.2 | Homo sapiens suppressor of variegation 4-20 homolog 2 (Drosophila) (SUV420H2), mRNA |
| 122 | UP | N90051 | 31543398 | NM_018367 | Homo sapiens phytoceramidase, alkaline (PHCA), mRNA |
| 123 | DOWN | N63635 | 31543400 | NM_002648 | Homo sapiens pim-1 oncogene (PIM1), mRNA |
| 124 | DOWN | AA443121 | 31543411 | NM_024310 | Homo sapiens pleckstrin homology domain containing, family F (with FYVE domain) member 1 |
| | | | | | (PLEKHF1), mRNA |
| 125 | UP | AA504615 | 31543656 | NM_006804 | Homo sapiens START domain containing 3 (STARD3), mRNA |
| 126 | DOWN | AA946732 | 31563510 | NM_015666.2 | Homo sapiens GTP binding protein 5 (putative) (GTPBP5), mRNA |
| 127 | DOWN | AA928656 | 31563516 | NM_006183.3 | Homo sapiens neurotensin (NTS), mRNA |
| 128 | DOWN | AA463461 | 31563520 | NM_024325 | Homo sapiens zinc finger protein 343 (ZNF343), mRNA |
| 129 | DOWN | AA291137 | 31581540 | NM_153689.3 | Homo sapiens hypothetical protein FLJ38973 (FLJ38973), mRNA |
| 130 | DOWN | AI276745 | 31881629 | NM_000956.2 | Homo sapiens prostaglandin E receptor 2 (subtype EP2), 53 kDa (PTGER2), mRNA |
| 131 | UP | AA885478 | 31982920 | NM_024749.2 | Homo sapiens hypothetical protein FLJ12505 (FLJ12505), mRNA |
| 132 | UP | AA777803 | 31982935 | NM_003901.2 | Homo sapiens sphingosine-1-phosphate lyase 1 (SGPL1), mRNA |
| 133 | DOWN | AA452257 | 32130532 | NM_181618 | Homo sapiens chemokine-like factor super family 5 (CKLFSF5), transcript variant 2, mRNA |
| 134 | UP | AA417744 | 32526889 | NM_152515.2 | Homo sapiens hypothetical protein FLJ40629 (FLJ40629), mRNA |
| 135 | DOWN | AA789328 | 32528262 | NM_003674 | Homo sapiens cyclin-dependent kinase (CDC2-like) 10 (CDK10), transcript variant 1, mRNA |
| 136 | DOWN | AA676660 | 32698685 | NM_014568.1 | Homo sapiens UDP-N-acetyl-alpha-D-galactosamine:polypeptide N- |
| | | | | | acetylgalactosaminyltransferase 5 (GalNAc-T5) (GALNT5), mRNA |
| 137 | DOWN | AA788805 | 32698729 | NM_020772 | Homo sapiens 82-kD FMRP Interacting Protein (182-FIP), mRNA |
| 138 | UP | N51614 | 33356147 | NM_005892 | Homo sapiens formin-like 1 (FMNL1), mRNA |
| 139 | DOWN | AI207748 | 33469927 | NM_024553.2 | Homo sapiens hypothetical protein FLJ20097 (FLJ20097), transcript variant 2, mRNA |
| 140 | DOWN | AA733195 | 33469972 | NM_016006 | Homo sapiens abhydrolase domain containing 5 (ABHD5), mRNA |
| 141 | DOWN | AA281733 | 33519477 | NM_001412 | Homo sapiens eukaryotic translation initiation factor 1A, X-linked (EIF1AX), mRNA |
| 142 | UP | H99824 | 33598951 | NM_006466.2 | Homo sapiens polymerase (RNA) III (DNA directed) polypeptide F, 39 kDa (POLR3F), mRNA |
| 143 | DOWN | H22826 | 33598967 | NM_005358 | Homo sapiens LIM domain 7 (LMO7), mRNA |
| 144 | UP | AA699808 | 33667066 | NM_014266.3 | Homo sapiens hematopoietic cell signal transducer (HCST), transcript variant 1, mRNA |
| 145 | DOWN | AA884159 | 33667114 | NM_182947.1 | Homo sapiens RAC/CDC42 exchange factor (GEFT), transcript variant 1, mRNA |
| 146 | UP | AA488868 | 33946290 | NM_024830.3 | Homo sapiens hypothetical protein FLJ12443 (FLJ12443), mRNA |
| 147 | UP | H83924 | 33946298 | NM_017922.2 | Homo sapiens PRP39 pre-mRNA processing factor 39 homolog (yeast) (PRPF39), mRNA |
| 148 | UP | AA905362 | 33946322 | NM_001177.3 | Homo sapiens ADP-ribosylation factor-like 1 (ARL1), mRNA |
| 149 | DOWN | AA984456 | 34101289 | NM_012306.2 | Homo sapiens Fas apoptotic inhibitory molecule 2 (FAIM2), mRNA |
| 150 | UP | AI203283 | 34147439 | NM_032726.2 | Homo sapiens phospholipase C, delta 4 (PLCD4), mRNA |
| 151 | DOWN | AA156247 | 34147494 | NM_004155 | Homo sapiens serine (or cysteine) proteinase inhibitor, clade 8 (ovalbumin), member 9 |
| | | | | | (SERPINB9), mRNA |
| 152 | DOWN | AA143609 | 34222091 | NM_001547.3 | Homo sapiens interferon-induced protein with tetratricopeptide repeats 2 (IFIT2), mRNA |
| 153 | UP | H79839 | 34222140 | NM_138774.2 | Homo sapiens chromosome 19 open reading frame 22 (C19orf22), mRNA |
| 154 | UP | AI024524 | 34222158 | NM_018275.3 | Homo sapiens hypothetical protein FLJ10925 (FLJ10925), mRNA |
| 155 | UP | AA703250 | 34222192 | NM_004697 | Homo sapiens PRP4 pre-mRNA processing factor 4 homolog (yeast) (PRPF4), mRNA |
| 156 | DOWN | N48345 | 34222238 | NM_173680 | Homo sapiens hypothetical protein MGC33584 (MGC33584), mRNA |
| 157 | UP | AA778089 | 34222384 | NM_020873.3 | Homo sapiens leucine rich repeat neuronal 1 (LRRN1), mRNA |
| 158 | UP | AI000878 | 34303919 | NM_024036.3 | Homo sapiens leucine rich repeat and fibronectin type III domain containing 4 (LRFN4), mRNA |
| 159 | DOWN | T47443 | 34335271 | NM_006404 | Homo sapiens protein C receptor, endothelial (EPCR) (PROCR), mRNA |
| 160 | UP | AA478794 | 34335395 | NM_032515 | Homo sapiens BCL2-related ovarian killer (BOK), mRNA |
| 161 | DOWN | AA128457 | 34452689 | NM_002928.2 | Homo sapiens regulator of G-protein signalling 16 (RGS16), mRNA |
| 162 | UP | AI671599 | 34485728 | NM_002556.2 | Homo sapiens oxysterol binding protein (OSBP), mRNA |
| 163 | DOWN | T89996 | 34734076 | NM_005438 | Homo sapiens FOS-like antigen 1 (FOSL1), mRNA |
| 164 | DOWN | N57657 | 34850073 | NM_016080 | Homo sapiens chromosome 17 open reading frame 25 (C17orf25), mRNA |
| 165 | DOWN | AI300766 | 34878715 | NM_003989.2 | Homo sapiens paired box gene 2 (PAX2), transcript variant d, mRNA |
| 166 | UP | AI291692 | 37059748 | NM_022771.3 | Homo sapiens TBC1 domain family, member 15 (TBC1D15), mRNA |
| 167 | UP | AA101088 | 37059777 | NM_017846.3 | Homo sapiens tRNA selenocysteine associated protein (SECP43), mRNA |
| 168 | UP | AA406206 | 37537711 | NM_022140 | Homo sapiens erythrocyte membrane protein band 4.1 like 4A (EPB41L4A), mRNA |
| 169 | UP | R01227 | 37577121 | NM_016021 | Homo sapiens ubiquitin-conjugating enzyme E2, J1 (UBC6 homolog, yeast) (UBE2J1), mRNA |
| 170 | UP | AA971179 | 37594445 | NM_138462.2 | Homo sapiens zinc finger, MYND domain containing 19 (ZMYND19), mRNA |
| 171 | DOWN | AA775791 | 37594454 | NM_032327.2 | Homo sapiens zinc finger, DHHC domain containing 16 (ZDHHC16), transcript variant 1, mRNA |
| 172 | DOWN | AI026814 | 37595546 | NM_002610.3 | Homo sapiens pyruvate dehydrogenase kinase, isoenzyme 1 (PDK1), nuclear gene encoding mitochondrial protein, mRNA |
| 173 | DOWN | AI016074 | 37655176 | NM_018355.2 | Homo sapiens zinc finger protein 415 (ZNF415), mRNA |
| 174 | DOWN | AA490605 | 37693523 | NM_014795 | Homo sapiens zinc finger homeobox 1b (ZFHX1B), mRNA |
| 175 | UP | AA670408 | 37704380 | NM_004048 | Homo sapiens beta-2-microglobulin (B2M), mRNA |
| 176 | UP | N53664 | 38016946 | NM_001735 | Homo sapiens complement component 5 (C5), mRNA |
| 177 | DOWN | N35020 | 38045936 | NM_016422 | Homo sapiens ring finger protein 141 (RNF141), mRNA |
| 178 | DOWN | W95118 | 38045937 | NM_014746 | Homo sapiens ring finger protein 144 (RNF144), mRNA |
| 179 | DOWN | AA101146 | 38176150 | NM_030650 | Homo sapiens KIAA1715 (KIAA1715), mRNA |
| 180 | UP | AA521473 | 38202212 | NM_006544.2 | Homo sapiens SEC10-like 1 (S. cerevisiae) (SEC10L1), mRNA |
| 181 | UP | AA454819 | 38257140 | NM_002746 | Homo sapiens mitogen-activated protein kinase 3 (MAPK3), mRNA |
| 182 | UP | AA917493 | 38348255 | NM_198468.1 | Homo sapiens chromosome 6 open reading frame 167 (C6orf167), mRNA |
| 183 | UP | AA885471 | 38348371 | NM_198537.1 | Homo sapiens FLJ44968 protein (FLJ44968), mRNA |
| 184 | DOWN | R00276 | 38454325 | NM_001775 | Homo sapiens CD38 antigen (p45) (CD38), mRNA |
| 185 | UP | H87275 | 38455387 | NM_001463 | Homo sapiens frizzled-related protein (FRZB), mRNA |
| 186 | DOWN | N30047 | 38504664 | NM_198845.1 | Homo sapiens sialic acid binding Ig-like lectin 6 (SIGLEC6), transcript variant 2, mRNA |
| 187 | DOWN | AA972628 | 38504668 | NM_198833.1 | Homo sapiens serine (or cysteine) proteinase inhibitor, clade B (ovalbumin), member 8 |
| | | | | | (SERPINB8), transcript variant 2, mRNA |
| 188 | DOWN | AA465228 | 38505210 | NM_198847.1 | Homo sapiens FLJ22794 protein (FLJ22794), transcript variant 2, mRNA |
| 189 | DOWN | AA599085 | 38569483 | NM_017641 | Homo sapiens kinesin family member 21A (KIF21A), mRNA |
| 190 | UP | N29860 | 38570089 | NM_004804 | Homo sapiens WD repeat domain 39 (WDR39), mRNA |
| 191 | UP | AI015577 | 38570147 | NM_032196.3 | Homo sapiens homolog of yeast INO80 (INO80), transcript variant 2, mRNA |
| 192 | DOWN | AI198629 | 38570157 | NM_004561.2 | Homo sapiens ovo-like 1(Drosophila) (OVOL1), mRNA |
| 193 | DOWN | AA016290 | 38683863 | NM_018703 | Homo sapiens retinoblastoma binding protein 6 (RBBP6), transcript variant 2, mRNA |
| 194 | DOWN | AI015297 | 39545576 | NM_016626.2 | Homo sapiens ring finger and KH domain containing 2 (RKHD2), mRNA |
| 195 | DOWN | AI347886 | 39725687 | NM_005002.3 | Homo sapiens NADH dehydrogenase (ubiquinone) 1 alpha subcomplex, 9, 39 kDa (NDUFA9), |
| | | | | | mRNA |
| 196 | DOWN | H19340 | 39753954 | NM_016641 | Homo sapiens membrane interacting protein of RGS16 (MIR16), mRNA |
| 197 | UP | AA903500 | 39777595 | NM_021809.4 | Homo sapiens TGFB-induced factor 2 (TALE family homeobox) (TGIF2), mRNA |
| 198 | DOWN | AA633873 | 39812393 | NM_031922.2 | Homo sapiens RALBP1 associated Eps domain containing 1 (REPS1), mRNA |
| 199 | DOWN | N45114 | 39841070 | NM_199005 | Homo sapiens zinc finger protein 322B (ZNF322B), mRNA |
| 200 | DOWN | AI334945 | 39995095 | NM_000960.3 | Homo sapiens prostaglandin I2 (prostacyclin) receptor (IP) (PTGIR), mRNA |
| 201 | DOWN | R50752 | 40068496 | NM_024091 | Homo sapiens hypothetical protein MGC5297 (MGC5297), mRNA |
| 202 | DOWN | AA778204 | 40217846 | NM_014014.2 | Homo sapiens activating signal cointegrator 1 complex subunit 3-like 1 (ASCC3L1), mRNA |
| 203 | DOWN | AA419251 | 40254449 | NM_003641 | Homo sapiens interferon induced transmembrane protein 1 (9-27) (IFITM1), mRNA |
| 204 | DOWN | AA909947 | 40255051 | NM_024726.3 | Homo sapiens IQ motif containing with AAA domain (IQCA), mRNA |
| 205 | DOWN | R60949 | 40255107 | NM_153013 | Homo sapiens hypothetical protein FLJ30596 (FLJ30596), mRNA |
| 206 | DOWN | H17954 | 40255204 | NM_175908 | Homo sapiens hypothetical protein LOC284296 (LOC284296), mRNA |
| 207 | UP | AI243595 | 40255224 | NM_022757.3 | Homo sapiens coiled-coil domain containing 14 (CCDC14), mRNA |
| 208 | DOWN | W69677 | 40255239 | NM_018045 | Homo sapiens hypothetical protein FLJ10276 (FLJ10276), mRNA |
| 209 | UP | AA256378 | 40288292 | NM_000361 | Homo sapiens thrombomodulin (THBD), mRNA |
| 210 | DOWN | N66469 | 40317633 | NM_199040.1 | Homo sapiens nudix (nucleoside diphosphate linked moiety X)-type motif 4 (NUDT4), transcript |
| | | | | | variant 2, mRNA |
| 211 | DOWN | W81159 | 40353763 | NM_199186.1 | Homo sapiens 2,3-bisphosphoglycerate mutase (BPGM), transcript variant 2, mRNA |
| 212 | DOWN | AA187938 | 40556362 | NM_016489 | Homo sapiens 5′-nucleotidase, cytosolic III (NT5C3), mRNA |
| 213 | DOWN | AA490096 | 40805869 | NM_199363.1 | Homo sapiens tumor protein D52-like 2 (TPD52L2), transcript variant 4, mRNA |
| 214 | DOWN | AI361530 | 40807490 | NM_001995.2 | Homo sapiens acyl-CoA synthetase long-chain family member 1 (ACSL1), mRNA |
| 215 | UP | R39454 | 41150548 | XM_373748.1 | PREDICTED: Homo sapiens hypothetical LOC388418 (LOC388418), mRNA |
| 216 | DOWN | W93315 | 41197059 | XM_374162 | PREDICTED: Homo sapiens hypothetical LOC389370 (LOC389370), mRNA |
| 217 | UP | W79511 | 41281472 | NM_014738 | Homo sapiens KIAA0195 gene product (KIAA0195), mRNA |
| 218 | UP | AA903137 | 41281490 | NM_014791.2 | Homo sapiens maternal embryonic leucine zipper kinase (MELK), mRNA |
| 219 | DOWN | AA908831 | 41327688 | NM_199513.1 | Homo sapiens chromosome 20 open reading frame 44 (C20orf44), transcript variant 3, mRNA |
| 220 | DOWN | R35665 | 41327737 | NM_005228 | Homo sapiens epidermal growth factor receptor (erythroblastic leukemia viral (v-erb-b) oncogene |
| | | | | | homolog, avian) (EGFR), transcript variant 1, mRNA |
| 221 | UP | AA479950 | 41327759 | NM_198531 | Homo sapiens ATPase, Class II, type 9B (ATP9B), mRNA |
| 222 | DOWN | N27177 | 41349453 | NM_201348.1 | Homo sapiens proline arginine-rich end leucine-rich repeat protein (PRELP), transcript variant 2, |
| | | | | | mRNA |
| 223 | DOWN | AA463931 | 41393564 | NM_014216 | Homo sapiens inositol 1,3,4-triphosphate 5/6 kinase (ITPK1), mRNA |
| 224 | DOWN | R26706 | 41872473 | NM_001987 | Homo sapiens ets variant gene 6 (TEL oncogene) (ETV6), mRNA |
| 225 | UP | AA133278 | 42476161 | NM_005398.3 | Homo sapiens protein phosphatase 1, regulatory (inhibitor) subunit 3C (PPP1R3C), mRNA |
| 226 | UP | AI131103 | 42544171 | NM_201999.1 | Homo sapiens E74-like factor 2 (ets domain transcription factor) (ELF2), transcript variant 1, |
| | | | | | mRNA |
| 227 | UP | R91087 | 42655888 | XM_375713.1 | PREDICTED: Homo sapiens hypothetical protein LOC284551 (LOC284551), mRNA |
| 228 | UP | AI217650 | 42656708 | XM_211843.2 | PREDICTED: Homo sapiens hypothetical protein LOC285326 (LOC285326), mRNA |
| 229 | UP | AA670305 | 42656764 | XM_117294.6 | PREDICTED: Homo sapiens hypothetical protein LOC200933 (LOC200933), mRNA |
| 230 | DOWN | AA975399 | 42658265 | XM_380099.1 | PREDICTED: Homo sapiens hypothetical LOC402476 (LOC402476), mRNA |
| 231 | UP | AA609312 | 42658886 | XM_114621 | PREDICTED: Homo sapiens similar to RIKEN cDNA 4930578I06 (LOC203076), mRNA |
| 232 | DOWN | AA923560 | 42716282 | NM_024598.2 | Homo sapiens hypothetical protein FLJ13154 (FLJ13154), mRNA |
| 233 | DOWN | AA173423 | 42734437 | NM_016623 | Homo sapiens family with sequence similarity 49, member B (FAM49B), mRNA |
| 234 | DOWN | AA452256 | 42741683 | NM_203350 | Homo sapiens zinc finger protein 265 (ZNF265), transcript variant 1, mRNA |
| 235 | DOWN | AA626335 | 44662835 | NM_014567.2 | Homo sapiens breast cancer anti-estrogen resistance 1 (BCAR1), mRNA |
| 236 | UP | AI383794 | 44771197 | NM_005788.1 | Homo sapiens HMT1 hnRNP methyltransferase-like 3 (S. cerevisiae) (HRMT1L3), mRNA |
| 237 | DOWN | AA909958 | 44917609 | NM_020882.1 | Homo sapiens KIAA1510 protein (KIAA1510), mRNA |
| 238 | DOWN | AA706974 | 44955890 | NM_001656 | Homo sapiens tripartite motif-containing 23 (TRIM23), transcript variant alpha, mRNA |
| 239 | DOWN | AA448711 | 4502326 | NM_001698 | Homo sapiens AU RNA binding protein/enoyl-Coenzyme A hydratase (AUH), nuclear gene |
| | | | | | encoding mitochondrial protein, mRNA |
| 240 | DOWN | AA676453 | 4502662 | NM_001774 | Homo sapiens CD37 antigen (CD37), mRNA |
| 241 | DOWN | AA112105 | 4503770 | NM_002027 | Homo sapiens farnesyltransferase, CAAX box, alpha (FNTA), mRNA |
| 242 | UP | N69049 | 4503832 | NM_003507 | Homo sapiens frizzled homolog 7 (Drosophila) (FZD7), mRNA |
| 243 | DOWN | N30428 | 4504132 | NM_000176 | Homo sapiens nuclear receptor subfamily 3, group C, member 1 (glucocorticoid receptor) |
| | | | | | (NR3C1), mRNA |
| 244 | UP | AI201184 | 4504142 | NM_000842.1 | Homo sapiens glutamate receptor, metabotropic 5 (GRM5), mRNA |
| 245 | UP | AA774833 | 4505664 | NM_000923.1 | Homo sapiens phosphodiesterase 4C, cAMP-specific (phosphodiesterase E1 dunce homolog, |
| | | | | | Drosophila) (PDE4C), mRNA |
| 246 | DOWN | T70056 | 4506558 | NM_002940 | Homo sapiens ATP-binding cassette, sub-family E (OABP), member 1 (ABCE1), mRNA |
| 247 | UP | T87622 | 4506782 | NM_003864.1 | Homo sapiens sin3-associated polypeptide, 30 kDa (SAP30), mRNA |
| 248 | UP | AA460823 | 4506910 | NM_000023 | Homo sapiens sarcoglycan, alpha (50 kDa dystrophin-associated glycoprotein) (SGCA), mRNA |
| 249 | UP | AI369312 | 4507106 | NM_003086.1 | Homo sapiens small nuclear RNA activating complex, polypeptide 4, 190 kDa (SNAPC4), mRNA |
| 250 | UP | AA936434 | 4507398 | NM_003199.1 | Homo sapiens transcription factor 4 (TCF4), mRNA |
| 251 | UP | N55274 | 4507464 | NM_003239 | Homo sapiens transforming growth factor, beta 3 (TGFB3), mRNA |
| 252 | DOWN | AI652954 | 4507474 | NM_000359.1 | Homo sapiens transglutaminase 1 (K polypeptide epidermal type I, protein-glutamine-gamma- |
| | | | | | glutamyltransferase) (TGM1), mRNA |
| 253 | DOWN | AA235388 | 4507552 | NM_003275 | Homo sapiens tropomodulin 1 (TMOD1), mRNA |
| 254 | UP | T50788 | 4507818 | NM_001076 | Homo sapiens UDP glycosyltransferase 2 family, polypeptide B15 (UGT2B15), mRNA |
| 255 | DOWN | AA426227 | 4507834 | NM_000373 | Homo sapiens uridine monophosphate synthetase (orotate phosphoribosyl transferase and |
| | | | | | orotidine-5′-decarboxylase) (UMPS), mRNA |
| 256 | UP | R62340 | 45505138 | NM_152309 | Homo sapiens phosphoinositide-3-kinase adaptor protein 1 (PIK3AP1), mRNA |
| 257 | DOWN | AA826373 | 45545410 | NM_205842.1 | Homo sapiens NCK-associated protein 1 (NCKAP1), transcript variant 2, mRNA |
| 258 | UP | AI382422 | 4557256 | NM_001115.1 | Homo sapiens adenylate cyclase 8 (brain) (ADCY8), mRNA |
| 259 | DOWN | AA055163 | 4557408 | NM_001232 | Homo sapiens calsequestrin 2 (cardiac muscle) (CASQ2), mRNA |
| 260 | DOWN | N24824 | 4557694 | NM_000222 | Homo sapiens v-kit Hardy-Zuckerman 4 feline sarcoma viral oncogene homolog (KIT), mRNA |
| 261 | DOWN | H11482 | 4557879 | NM_000416 | Homo sapiens interferon gamma receptor 1 (IFNGR1), mRNA |
| 262 | DOWN | N63943 | 4557893 | NM_000239 | Homo sapiens lysozyme (renal amyloidosis) (LYZ), mRNA |
| 263 | UP | T70999 | 45827761 | NM_019075.2 | Homo sapiens UDP glycosyltransferase 1 family, polypeptide A10 (UGT1A10), mRNA |
| 264 | DOWN | N21237 | 45935384 | NM_015278 | Homo sapiens SAM and SH3 domain containing 1 (SASH1), mRNA |
| 265 | DOWN | AA916546 | 46048219 | NM_031949.3 | Homo sapiens tubulin tyrosine ligase-like family, member 2 (TTLL2), mRNA |
| 266 | UP | AA705999 | 46195722 | NM_015659 | Homo sapiens ribosomal L1 domain containing 1 (RSL1D1), mRNA |
| 267 | DOWN | AA426025 | 46249354 | NM_199332 | Homo sapiens homer homolog 2(Drosophila) (HOMER2), transcript variant 4, mRNA |
| 268 | DOWN | AA521439 | 46255067 | NM_206930.1 | Homo sapiens synaptotagmin-like 2 (SYTL2), transcript variant f, mRNA |
| 269 | UP | AI361112 | 46358421 | NM_002429.3 | Homo sapiens matrix metalloproteinase 19 (MMP19), transcript variant rasi-1, mRNA |
| 270 | DOWN | N58163 | 46397354 | NM_024345 | Homo sapiens WD repeat domain 32 (WDR32), mRNA |
| 271 | UP | AA971725 | 46397374 | NM_018150.2 | Homo sapiens hypothetical protein FLJ10597 (FLJ10597), mRNA |
| 272 | DOWN | AA884901 | 46409491 | NM_207434.1 | Homo sapiens FLJ46363 protein (FLJ46363), mRNA |
| 273 | DOWN | AA489017 | 46877103 | NM_003367 | Homo sapiens upstream transcription factor 2, c-fos interacting (USF2), transcript variant 1, |
| | | | | | mRNA |
| 274 | UP | AA394136 | 47078232 | NM_212503 | Homo sapiens PCTAIRE protein kinase 3 (PCTK3), transcript variant 1, mRNA |
| 275 | UP | AA005215 | 47157324 | NM_212539.1 | Homo sapiens protein kinase C, delta (PRKCD), transcript variant 2, mRNA |
| 276 | DOWN | AA683550 | 4755143 | NM_001569 | Homo sapiens interleukin-1 receptor-associated kinase 1 (IRAK1), mRNA |
| 277 | DOWN | AA027049 | 4757889 | NM_004337 | Homo sapiens chromosome 8 open reading frame 1 (C8orf1), mRNA |
| 278 | DOWN | AA479196 | 4758487 | NM_004128 | Homo sapiens general transcription factor IIF, polypeptide 2 (30 kD subunit) (GTF2F2), mRNA |
| 279 | DOWN | AA877255 | 4758609 | NM_001572 | Homo sapiens interferon regulatory factor 7 (IRF7), transcript variant a, mRNA |
| 280 | UP | AA425316 | 47778926 | NM_001001336 | Homo sapiens cytochrome b5 reductase b5R.2 (CYB5R2), transcript variant 2, mRNA |
| 281 | UP | AA425754 | 47933378 | NM_003827 | Homo sapiens N-ethylmaleimide-sensitive factor attachment protein, alpha (NAPA), mRNA |
| 282 | DOWN | AA044390 | 48255967 | NM_001001521 | Homo sapiens UDP-glucose pyrophosphorylase 2 (UGP2), transcript variant 2, mRNA |
| 283 | UP | AI016760 | 4826947 | NM_005044.1 | Homo sapiens protein kinase, X-linked (PRKX), mRNA |
| 284 | DOWN | W32509 | 48375181 | NM_012121 | Homo sapiens CDC42 effector protein (Rho GTPase binding) 4 (CDC42EP4), mRNA |
| 285 | DOWN | H22936 | 48675821 | NM_145257 | Homo sapiens LOC126731 (LOC126731), mRNA |
| 286 | UP | AI028325 | 50086627 | NM_145053.3 | Homo sapiens hypothetical protein MGC20470 (MGC20470), mRNA |
| 287 | UP | H43617 | 5031650 | NM_005671.1 | Homo sapiens reproduction 8 (D8S2298E), mRNA |
| 288 | UP | N26562 | 5031912 | NM_005511 | Homo sapiens melan-A (MLANA), mRNA |
| 289 | DOWN | N63628 | 50355983 | NM_024790 | Homo sapiens centrosome spindle pole associated protein (CSPP), mRNA |
| 290 | DOWN | AI015246 | 50659063 | NM_173360.2 | Homo sapiens spermatogenesis associated 9 (SPATA9), transcript variant 2, mRNA |
| 291 | DOWN | W59987 | 50726964 | NM_013392 | Homo sapiens nuclear receptor binding protein (NRBP), mRNA |
| 292 | DOWN | R34323 | 50843836 | NM_018071 | Homo sapiens hypothetical protein FLJ10357 (FLJ10357), mRNA |
| 293 | DOWN | AI016456 | 50897271 | NM_001002910.1 | Homo sapiens cytochrome P450, family 2, subfamily D, polypeptide 7 pseudogene 1 |
| | | | | | (CYP2D7P1), mRNA |
| 294 | DOWN | H23444 | 50962798 | NM_213609 | Homo sapiens family with sequence similarity 19 (chemokine (C—C motif)-like), member A1 |
| | | | | | (FAM19A1), mRNA |
| 295 | DOWN | AA598573 | 51100963 | NM_025154 | Homo sapiens unc-84 homolog A (C. elegans) (UNC84A), mRNA |
| 296 | DOWN | W81563 | 51173146 | NM_000232 | Homo sapiens sarcoglycan, beta (43 kDa dystrophin-associated glycoprotein) (SGCB), mRNA |
| 297 | DOWN | AI014441 | 51173747 | NM_001003397.1 | Homo sapiens tumor protein D52-like 1 (TPD52L1), transcript variant 4, mRNA |
| 298 | DOWN | AA989225 | 51243060 | NM_014507.2 | Homo sapiens malonyl-CoA: acyl carrier protein transacylase, mitochondrial (MT), nuclear gene |
| | | | | | encoding mitochondrial protein, transcript variant 2, mRNA |
| 299 | UP | R74169 | 51458700 | XM_496405 | PREDICTED: Homo sapiens similar to D(1B) dopamine receptor (D(5) dopamine receptor) |
| | | | | | (D1beta dopamine receptor) (LOC440684), mRNA |
| 300 | DOWN | R51886 | 51460531 | XM_039676 | PREDICTED: Homo sapiens KIAA1240 protein (KIAA1240), mRNA |
| 301 | DOWN | AA954477 | 51464134 | XM_379262.2 | PREDICTED: Homo sapiens hypothetical LOC401124 (LOC401124), mRNA |
| 302 | UP | AA862946 | 51464169 | XM_373030.3 | PREDICTED: Homo sapiens hypothetical protein LOC285556 (LOC285556), mRNA |
| 303 | DOWN | AA026276 | 51464481 | XM_374078 | PREDICTED: Homo sapiens hypothetical protein LOC285548 (LOC285548), mRNA |
| 304 | UP | AA287347 | 51465304 | XM_371848.3 | PREDICTED: Homo sapiens chromosome 6 open reading frame 115 (C6orf115), mRNA |
| 305 | DOWN | AI003036 | 51465523 | XR_000208.3 | PREDICTED: Homo sapiens zinc finger protein 204 (ZNF204), misc RNA |
| 306 | DOWN | AA482282 | 51466733 | XM_372035.2 | PREDICTED: Homo sapiens hypothetical LOC389643 (LOC389643), mRNA |
| 307 | UP | AI311125 | 51467635 | XM_499165.1 | PREDICTED: Homo sapiens hypothetical gene supported by AK024177 (LOC441468), mRNA |
| 308 | UP | AI301143 | 51470756 | XM_291947.5 | PREDICTED: Homo sapiens hephaestin-like (LOC341208), mRNA |
| 309 | DOWN | AA904604 | 51470865 | XM_096472.2 | PREDICTED: Homo sapiens similar to RIKEN cDNA 1700029I15 (LOC143678), mRNA |
| 310 | UP | AA402879 | 51471183 | XM_495908 | PREDICTED: Homo sapiens similar to DEAD/H (Asp-Glu-Ala-Asp/His) box polypeptide 11; yeast |
| | | | | | CHL1 homolog; DEAD/H box-11 (CHL1-related helicase gene-1); DEAD/H (Asp-Glu-Ala- |
| | | | | | Asp/His) box polypeptide 11 (S. cerevisiae CHL1-like helicase) (LOC440081), mRNA |
| 311 | UP | H71854 | 51471269 | XM_378389 | PREDICTED: Homo sapiens hypothetical LOC400084 (LOC400084), mRNA |
| 312 | UP | AI018012 | 51471411 | XM_498558.1 | PREDICTED: Homo sapiens LOC440127 (LOC440127), mRNA |
| 313 | DOWN | AA682634 | 51471757 | XM_113763.6 | PREDICTED: Homo sapiens chromosome 14 open reading frame 125 (C14orf125), mRNA |
| 314 | UP | AI023820 | 51472346 | XM_496061.1 | PREDICTED: Homo sapiens similar to FLJ44796 protein (LOC440264), mRNA |
| 315 | DOWN | AA429886 | 51472865 | XM_370965 | PREDICTED: Homo sapiens similar to hypothetical protein BC011981 (LOC388242), mRNA |
| 316 | DOWN | AA936435 | 51472989 | XM_373685.3 | PREDICTED: Homo sapiens hypothetical LOC388276 (LOC388276), mRNA |
| 317 | UP | R39110 | 51474260 | XM_375456.2 | PREDICTED: Homo sapiens hypothetical protein DKFZp761G2113 (DKFZp761G2113), mRNA |
| 318 | DOWN | AA682513 | 51474376 | XM_294894.2 | PREDICTED: Homo sapiens hypothetical LOC339281 (LOC339281), mRNA |
| 319 | UP | AA219282 | 51474582 | XM_371116 | PREDICTED, Homo sapiens myosin VB (MYO5B), mRNA |
| 320 | DOWN | AA131794 | 51474602 | XM_378758 | PREDICTED: Homo sapiens hypothetical LOC400660 (LOC400660), mRNA |
| 321 | DOWN | AA446005 | 51475168 | XM_045421 | PREDICTED: Homo sapiens chromosome 20 open reading frame 194 (C20orf194), mRNA |
| 322 | DOWN | AA873159 | 51944963 | NM_001645 | Homo sapiens apolipoprotein C-I (APOC1), mRNA |
| 323 | UP | N71959 | 52138573 | NM_031465.2 | Homo sapiens hypothetical protein MGC13204 (MGC13204), mRNA |
| 324 | DOWN | W47387 | 52630437 | NM_005665 | Homo sapiens ecotropic viral integration site 5 (EVI5), mRNA |
| 325 | UP | AA164782 | 52694670 | NM_031461 | Homo sapiens cysteine-rich secretory protein LCCL domain containing 1 (CRISPLD1), mRNA |
| 326 | UP | AA706968 | 53729321 | NM_001005414 | Homo sapiens ZW10 interactor (ZWINT), transcript variant 4, mRNA |
| 327 | DOWN | W69269 | 53729347 | NM_007183.2 | Homo sapiens plakophilin 3 (PKP3), mRNA |
| 328 | DOWN | AA885140 | 53759068 | NM_017923.2 | Homo sapiens membrane-associated ring finger (C3HC4) 1 (MARCH1), mRNA |
| 329 | DOWN | T54342 | 53832029 | NM_016433 | Homo sapiens glycolipid transfer protein (GLTP), mRNA |
| 330 | DOWN | AA971634 | 53832030 | NM_004489.4 | Homo sapiens G protein pathway suppressor 2 (GPS2), mRNA |
| 331 | DOWN | AA644224 | 54112402 | NM_017780.2 | Homo sapiens chromodomain helicase DNA binding protein 7 (CHD7), mRNA |
| 332 | DOWN | AA620960 | 54262138 | NM_053064.2 | Homo sapiens guanine nucleotide binding protein (G protein), gamma 2 (GNG2), mRNA |
| 333 | UP | N38852 | 54262144 | NM_198484.2 | Homo sapiens zinc finger protein 621 (ZNF621), mRNA |
| 334 | DOWN | AA974801 | 5453596 | NM_006135.1 | Homo sapiens capping protein (actin filament) muscle Z-line, alpha 1 (CAPZA1), mRNA |
| 335 | DOWN | AA991931 | 5454045 | NM_006323.1 | Homo sapiens SEC24 related gene family, member B (S. cerevisiae) (SEC24B), mRNA |
| 336 | DOWN | AA279990 | 5454101 | NM_006342 | Homo sapiens transforming, acidic coiled-coil containing protein 3 (TACC3), mRNA |
| 337 | DOWN | R68828 | 54607076 | NM_023016.2 | Homo sapiens chromosome 2 open reading frame 26 (C2orf26), mRNA |
| 338 | UP | H18640 | 54792076 | NM_001006635.1 | Homo sapiens metaxin 2 (MTX2), transcript variant 2, mRNA |
| 339 | UP | AA779733 | 55741652 | NM_020931.1 | Homo sapiens KIAA1586 (KIAA1586), mRNA |
| 340 | DOWN | AA425435 | 55742785 | NM_020754 | Homo sapiens Cdc42 GTPase-activating protein (CDGAP), mRNA |
| 341 | UP | R23251 | 55743087 | NM_001006942 | Homo sapiens asparagine-linked glycosylation 3 homolog (yeast, alpha-1,3- |
| | | | | | mannosyltransferase) (ALG3), transcript variant 2, mRNA |
| 342 | DOWN | N34426 | 55749768 | NM_020943 | Homo sapiens KIAA1604 protein (KIAA1604), mRNA |
| 343 | UP | R91516 | 55953105 | NM_001007267 | Homo sapiens phospholipase A2 receptor 1, 180 kDa (PLA2R1), transcript variant 2, mRNA |
| 344 | DOWN | AA232647 | 56118213 | NM_007146 | Homo sapiens zinc finger protein 161 (ZNF161), mRNA |
| 345 | DOWN | AA878455 | 56243589 | NM_018383.3 | Homo sapiens WD repeat domain 33 (WDR33), transcript variant 1, mRNA |
| 346 | DOWN | R67903 | 56549112 | NM_016216 | Homo sapiens debranching enzyme homolog 1 (S. cerevisiae) (DBR1), mRNA |
| 347 | UP | AA709322 | 56550119 | NM_017619.3 | Homo sapiens RNA-binding region (RNP1, RRM) containing 3 (RNPC3), mRNA |
| 348 | UP | AA433885 | 56606138 | NM_001008234 | Homo sapiens hypothetical gene supported by BC036588 (LOC400657), mRNA |
| 349 | DOWN | AA443302 | 56676394 | NM_005168 | Homo sapiens Rho family GTPase 3 (RND3), mRNA |
| 350 | DOWN | AA421270 | 56699479 | NM_015622 | Homo sapiens chromosome 7 open reading frame 28A (C7orf28A), mRNA |
| 351 | UP | AA112057 | 56711247 | NM_015137 | Homo sapiens KIAA0143 protein (KIAA0143), mRNA |
| 352 | UP | R55763 | 56711259 | NM_020819 | Homo sapiens KIAA1411 (KIAA1411), mRNA |
| 353 | DOWN | AA936454 | 56711311 | NM_018555.4 | Homo sapiens zinc finger protein 331 (ZNF331), mRNA |
| 354 | DOWN | AA621302 | 56711315 | NM_173551 | Homo sapiens sterile alpha motif domain containing 6 (SAMD6), mRNA |
| 355 | UP | W90716 | 56788369 | NM_012455.2 | Homo sapiens pleckstrin and Sec7 domain containing 4 (PSD4), mRNA |
| 356 | DOWN | AA772502 | 56788376 | NM_001008564 | Homo sapiens nucleoporin like 1 (NUPL1), transcript variant 2, mRNA |
| 357 | UP | AA215643 | 57242804 | NM_001008744.1 | Homo sapiens tyrosyl-DNA phosphodiesterase 1 (TDP1), transcript variant 2, mRNA |
| 358 | UP | AA905085 | 57770637 | NR_002187.1 | Homo sapiens hypothetical protein LOC286016 (LOC286016) on chromosome 7 |
| 359 | DOWN | AA171735 | 57863270 | NM_015058 | Homo sapiens KIAA0564 protein (KIAA0564), transcript variant 1, mRNA |
| 360 | DOWN | R49243 | 57863758 | NM_001009899 | Homo sapiens KIAA2018 (KIAA2018), mRNA |
| 361 | DOWN | W53016 | 58219791 | NM_001010942 | Homo sapiens RAP1B, member of RAS oncogene family (RAP1B), transcript variant 2, mRNA |
| 362 | UP | AA169801 | 58331110 | NM_019046.1 | Homo sapiens ankyrin repeat domain 16 (ANKRD16), transcript variant 1, mRNA |
| 363 | UP | R94601 | 58331194 | NM_015436 | Homo sapiens ring finger and CHY zinc finger domain containing 1 (RCHY1), transcript variant |
| | | | | | 1, mRNA |
| 364 | DOWN | AA148683 | 58532586 | NM_001002860 | Homo sapiens BTB (POZ) domain containing 7 (BTBD7), transcript variant 1, mRNA |
| 365 | UP | W45589 | 5902065 | NM_007042 | Homo sapiens ribonuclease P 14 kDa subunit (RPP14), mRNA |
| 366 | UP | AA985471 | 6005831 | NM_007221.1 | Homo sapiens polyamine-modulated factor 1 (PMF1), mRNA |
| 367 | DOWN | AA436187 | 6006013 | NM_000632 | Homo sapiens integrin, alpha M (complement component receptor 3, alpha; also known as |
| | | | | | CD11b (p170), macrophage antigen alpha polypeptide) (ITGAM), mRNA |
| 368 | DOWN | AA932441 | 6042195 | NM_003793.2 | Homo sapiens cathepsin F (CTSF), mRNA |
| 369 | DOWN | AA489033 | 61742163 | NM_001009894.2 | Homo sapiens hypothetical protein DKFZp434N2030 (DKFZp434N2030), mRNA |
| 370 | DOWN | T57765 | 61743953 | NM_001620 | Homo sapiens AHNAK nucleoprotein (desmoyokin) (AHNAK), transcript variant 1, mRNA |
| 371 | DOWN | AI338894 | 62241047 | NM_013231.4 | Homo sapiens fibronectin leucine rich transmembrane protein 2 (FLRT2), mRNA |
| 372 | DOWN | N48319 | 62243893 | NM_003567.2 | Homo sapiens breast cancer anti-estrogen resistance 3 (BCAR3), mRNA |
| 373 | UP | AA703019 | 62865646 | NM_016530.2 | Homo sapiens RAB8B, member RAS oncogene family (RAB8B), mRNA |
| 374 | UP | AA777584 | 63175651 | NM_153232.3 | Homo sapiens CREBBP/EP300 inhibitor 2 (CRI2), mRNA |
| 375 | UP | R51015 | 64085120 | NM_000369 | Homo sapiens thyroid stimulating hormone receptor (TSHR), transcript variant 1, mRNA |
| 376 | DOWN | AA400393 | 64276807 | NM_197941 | Homo sapiens a disintegrin-like and metalloprotease (reprolysin type) with thrombospondin type |
| | | | | | 1 motif, 6 (ADAMTS6), mRNA |
| 377 | DOWN | AA666096 | 64762483 | NM_001018055.1 | Homo sapiens chromosome X open reading frame 53 (CXorf53), transcript variant 2, mRNA |
| 378 | DOWN | AA903175 | 6679051 | NM_007360.1 | Homo sapiens killer cell lectin-like receptor subfamily K, member 1 (KLRK1), mRNA |
| 379 | UP | AA708940 | 6912481 | NM_012318.1 | Homo sapiens leucine zipper-EF-hand containing transmembrane protein 1 (LETM1), mRNA |
| 380 | DOWN | AA283007 | 6996012 | NM_006144 | Homo sapiens granzyme A (granzyme 1, cytotoxic T-lymphocyte-associated serine esterase 3) |
| | | | | | (GZMA), mRNA |
| 381 | DOWN | AA995904 | 7019370 | NM_013342.1 | Homo sapiens TCF3 (E2A) fusion partner (in childhood Leukemia) (TFPT), mRNA |
| 382 | UP | AI025113 | 7019492 | NM_013284.1 | Homo sapiens polymerase (DNA directed), mu (POLM), mRNA |
| 383 | DOWN | AA918328 | 7657045 | NM_014501.1 | Homo sapiens ubiquitin-conjugating enzyme E2S (UBE2S), mRNA |
| 384 | DOWN | R35079 | 7657674 | NM_014232 | Homo sapiens vesicle-associated membrane protein 2 (synaptobrevin 2) (VAMP2), mRNA |
| 385 | DOWN | AA427857 | 7661605 | NM_015658.1 | Homo sapiens DKFZP564C186 protein (DKFZP564C186), mRNA |
| 386 | DOWN | T77847 | 7661669 | NM_015677 | Homo sapiens SH3 domain containing, Ysc84-like 1 (S. cerevisiae) (SH3YL1), mRNA |
| 387 | DOWN | AA487265 | 7661907 | NM_014752 | Homo sapiens signal peptidase complex subunit 2 homolog (S. cerevisiae) (SPCS2), mRNA |
| 388 | UP | AA953508 | 7662121 | NM_015559.1 | Homo sapiens SET binding protein 1 (SETBP1), mRNA |
| 389 | DOWN | N56973 | 7662213 | NM_014789 | Homo sapiens zinc finger protein 623 (ZNF623), mRNA |
| 390 | DOWN | AA446839 | 7669480 | NM_004052 | Homo sapiens BCL2/adenovirus E1B 19 kDa interacting protein 3 (BNIP3), nuclear gene |
| | | | | | encoding mitochondrial protein, mRNA |
| 391 | DOWN | T55608 | 7705786 | NM_016022 | Homo sapiens anterior pharynx defective 1 homolog A (C. elegans) (APH1A), mRNA |
| 392 | DOWN | AI341526 | 7710111 | NM_015831.1 | Homo sapiens acetylcholinesterase (YT blood group) (ACHE), transcript variant E4-E5, mRNA |
| 393 | DOWN | H20204 | 8922076 | NM_018702 | Homo sapiens adenosine deaminase, RNA-specific, B2 (RED2 homolog rat) (ADARB2), mRNA |
| 394 | UP | R53470 | 8922630 | NM_018199.1 | Homo sapiens chromosome 14 open reading frame 114 (C14orf114), mRNA |
| 395 | DOWN | AA599183 | 8922678 | NM_018225 | Homo sapiens smu-1 suppressor of mec-8 and unc-52 homolog (C. elegans) (SMU1), mRNA |
| 396 | DOWN | AA905628 | 8922691 | NM_018231.1 | Homo sapiens amino acid transporter (FLJ10815), mRNA |
| 397 | DOWN | AA127861 | 8923142 | NM_017686 | Homo sapiens ganglioside induced differentiation associated protein 2 (GDAP2), mRNA |
| 398 | UP | AA063624 | 8923397 | NM_017816 | Homo sapiens hypothetical protein FLJ20425 (LYAR), mRNA |
| 399 | UP | T82457 | 8923474 | NM_017853.1 | Homo sapiens thioredoxin-like 4B (TXNL4B), mRNA |
| 400 | DOWN | AA960957 | 8923753 | NM_018401.1 | Homo sapiens serine/threonine kinase 32B (STK32B), mRNA |
| 401 | DOWN | AI184305 | 9945331 | NM_015675.1 | Homo sapiens growth arrest and DNA-damage-inducible, beta (GADD45B), mRNA |

TABLE 3
List of mRNAs differentially expressed in asthma subjects

| SEQ ID NO. | Regulation | Accession on chip |
|---|---|---|
| 402 | UP | AA913864 |
| 403 | DOWN | AI732176 |
| 404 | DOWN | AI732945 |
| 405 | DOWN | AI733047 |
| 406 | DOWN | AI733078 |
| 407 | UP | AI733081 |
| 408 | DOWN | AI733117 |
| 409 | DOWN | AI733136 |
| 410 | DOWN | AI733140 |
| 411 | DOWN | AI733203 |
| 412 | DOWN | AI733226 |
| 413 | DOWN | AI733311 |
| 414 | DOWN | AI733333 |
| 415 | UP | AI733348 |
| 416 | DOWN | AI733440 |
| 417 | DOWN | AI733643 |
| 418 | UP | AI791140 |
| 419 | DOWN | AI820648 |
| 420 | DOWN | T99030 |
| 421 | DOWN | AA005292 |
| 422 | DOWN | AA035344 |
| 423 | DOWN | AA041254 |
| 424 | UP | AA045253 |
| 425 | UP | AA045527 |
| 426 | UP | AA069452 |
| 427 | UP | AA101771 |
| 428 | UP | AA115749 |
| 429 | DOWN | AA136071 |
| 430 | DOWN | AA150107 |
| 431 | DOWN | AA173109 |
| 432 | DOWN | AA194070 |
| 433 | DOWN | AA210707 |
| 434 | DOWN | AA214559 |
| 435 | DOWN | AA251129 |
| 436 | DOWN | AA251363 |
| 437 | UP | AA256073 |
| 438 | UP | AA282292 |
| 439 | UP | AA284067 |
| 440 | UP | AA291138 |
| 441 | UP | AA293192 |
| 442 | DOWN | AA416775 |
| 443 | DOWN | AA417618 |
| 444 | UP | AA421484 |
| 445 | DOWN | AA423977 |
| 446 | DOWN | AA424944 |
| 447 | DOWN | AA426344 |
| 448 | DOWN | AA431761 |
| 449 | DOWN | AA446013 |
| 450 | DOWN | AA446316 |
| 451 | DOWN | AA448192 |
| 452 | UP | AA452807 |
| 453 | DOWN | AA459358 |
| 454 | DOWN | AA461501 |
| 455 | DOWN | AA461534 |
| 456 | DOWN | AA463625 |
| 457 | DOWN | AA479912 |
| 458 | UP | AA481412 |
| 459 | DOWN | AA481492 |
| 460 | DOWN | AA489061 |
| 461 | DOWN | AA489200 |
| 462 | DOWN | AA489218 |
| 463 | DOWN | AA489652 |
| 464 | UP | AA489697 |
| 465 | UP | AA490225 |
| 466 | DOWN | AA490892 |
| 467 | UP | AA491285 |
| 468 | UP | AA496936 |
| 469 | UP | AA504457 |
| 470 | DOWN | AA521339 |
| 471 | DOWN | AA599376 |
| 472 | DOWN | AA600238 |
| 473 | DOWN | AA609215 |
| 474 | DOWN | AA620598 |
| 475 | UP | AA620955 |
| 476 | UP | AA626256 |
| 477 | UP | AA626311 |
| 478 | UP | AA628243 |
| 479 | DOWN | AA629901 |
| 480 | DOWN | AA629903 |
| 481 | DOWN | AA629991 |
| 482 | UP | AA630208 |
| 483 | UP | AA630305 |
| 484 | UP | AA630371 |
| 485 | DOWN | AA644587 |
| 486 | UP | AA663920 |
| 487 | UP | AA668647 |
| 488 | UP | AA670270 |
| 489 | UP | AA676246 |
| 490 | UP | AA676259 |
| 491 | DOWN | AA676713 |
| 492 | UP | AA677246 |
| 493 | DOWN | AA677461 |
| 494 | DOWN | AA677671 |
| 495 | DOWN | AA677798 |
| 496 | DOWN | AA678047 |
| 497 | DOWN | AA680221 |
| 498 | DOWN | AA682248 |
| 499 | DOWN | AA682505 |
| 500 | DOWN | AA693513 |
| 501 | DOWN | AA699850 |
| 502 | DOWN | AA700108 |
| 503 | UP | AA700433 |
| 504 | UP | AA702723 |
| 505 | DOWN | AA704322 |
| 506 | UP | AA704896 |
| 507 | UP | AA704981 |
| 508 | DOWN | AA705306 |
| 509 | DOWN | AA705431 |
| 510 | DOWN | AA706864 |
| 511 | DOWN | AA706966 |
| 512 | DOWN | AA708157 |
| 513 | DOWN | AA732827 |
| 514 | DOWN | AA773037 |
| 515 | DOWN | AA775574 |
| 516 | UP | AA775606 |
| 517 | DOWN | AA776813 |
| 518 | DOWN | AA776828 |
| 519 | UP | AA776844 |
| 520 | DOWN | AA778551 |
| 521 | UP | AA779460 |
| 522 | UP | AA779843 |
| 523 | DOWN | AA779865 |
| 524 | UP | AA780042 |
| 525 | DOWN | AA827297 |
| 526 | DOWN | AA858162 |
| 527 | UP | AA864787 |
| 528 | DOWN | AA872282 |
| 529 | DOWN | AA878939 |
| 530 | DOWN | AA879423 |
| 531 | DOWN | AA883127 |
| 532 | DOWN | AA883353 |
| 533 | UP | AA883755 |
| 534 | DOWN | AA884326 |
| 535 | DOWN | AA884399 |
| 536 | DOWN | AA884412 |
| 537 | DOWN | AA884428 |
| 538 | UP | AA886178 |
| 539 | DOWN | AA886414 |
| 540 | UP | AA886892 |
| 541 | DOWN | AA887525 |
| 542 | DOWN | AA889041 |
| 543 | DOWN | AA890067 |
| 544 | DOWN | AA890098 |
| 545 | DOWN | AA894855 |
| 546 | DOWN | AA904777 |
| 547 | UP | AA905098 |
| 548 | DOWN | AA905113 |
| 549 | DOWN | AA905145 |
| 550 | UP | AA905331 |
| 551 | DOWN | AA905968 |
| 552 | DOWN | AA906209 |
| 553 | UP | AA907727 |
| 554 | DOWN | AA908678 |
| 555 | DOWN | AA909664 |
| 556 | DOWN | AA909688 |
| 557 | DOWN | AA909982 |
| 558 | UP | AA910454 |
| 559 | UP | AA913408 |
| 560 | DOWN | AA917044 |
| 561 | DOWN | AA917854 |
| 562 | UP | AA918191 |
| 563 | DOWN | AA918197 |
| 564 | DOWN | AA919064 |
| 565 | DOWN | AA919087 |
| 566 | DOWN | AA919125 |
| 567 | UP | AA921759 |
| 568 | UP | AA921894 |
| 569 | DOWN | AA922734 |
| 570 | DOWN | AA922858 |
| 571 | UP | AA923375 |
| 572 | DOWN | AA927170 |
| 573 | DOWN | AA927221 |
| 574 | DOWN | AA927646 |
| 575 | DOWN | AA927904 |
| 576 | DOWN | AA928710 |
| 577 | UP | AA933076 |
| 578 | DOWN | AA935909 |
| 579 | DOWN | AA938927 |
| 580 | DOWN | AA939238 |
| 581 | DOWN | AA948144 |
| 582 | UP | AA952874 |
| 583 | DOWN | AA953066 |
| 584 | DOWN | AA954771 |
| 585 | DOWN | AA960970 |
| 586 | DOWN | AA961109 |
| 587 | DOWN | AA962272 |
| 588 | DOWN | AA968436 |
| 589 | DOWN | AA970022 |
| 590 | DOWN | AA970023 |
| 591 | DOWN | AA970083 |
| 592 | DOWN | AA970307 |
| 593 | DOWN | AA970505 |
| 594 | DOWN | AA970843 |
| 595 | UP | AA971014 |
| 596 | DOWN | AA971493 |
| 597 | UP | AA972930 |
| 598 | DOWN | AA973496 |
| 599 | UP | AA973750 |
| 600 | DOWN | AA975103 |
| 601 | DOWN | AA975413 |
| 602 | UP | AA975507 |
| 603 | DOWN | AA976049 |
| 604 | UP | AA976649 |
| 605 | DOWN | AA977487 |
| 606 | DOWN | AA977598 |
| 607 | DOWN | AA977618 |
| 608 | DOWN | AA983875 |
| 609 | DOWN | AA984678 |
| 610 | DOWN | AA984894 |
| 611 | DOWN | AA987928 |
| 612 | DOWN | AA988036 |
| 613 | DOWN | AA989432 |
| 614 | DOWN | AA991912 |
| 615 | DOWN | AA993923 |
| 616 | DOWN | AA993948 |
| 617 | UP | AA993998 |
| 618 | UP | AA994520 |
| 619 | DOWN | AA994735 |
| 620 | DOWN | AI003528 |
| 621 | DOWN | AI004001 |
| 622 | UP | AI005126 |
| 623 | UP | AI005270 |
| 624 | DOWN | AI005275 |
| 625 | DOWN | AI015751 |
| 626 | UP | AI015890 |
| 627 | DOWN | AI016090 |
| 628 | DOWN | AI017618 |
| 629 | DOWN | AI024808 |
| 630 | DOWN | AI025349 |
| 631 | UP | AI028328 |
| 632 | UP | AI028395 |
| 633 | UP | AI033510 |
| 634 | UP | AI079530 |
| 635 | UP | AI091505 |
| 636 | UP | AI126052 |
| 637 | UP | AI126464 |
| 638 | UP | AI127087 |
| 639 | DOWN | AI141063 |
| 640 | DOWN | AI146610 |
| 641 | DOWN | AI147867 |
| 642 | UP | AI151218 |
| 643 | UP | AI183541 |
| 644 | UP | AI184207 |
| 645 | UP | AI190157 |
| 646 | DOWN | AI190728 |
| 647 | UP | AI198877 |
| 648 | UP | AI204175 |
| 649 | UP | AI204532 |
| 650 | UP | AI204957 |
| 651 | UP | AI207146 |
| 652 | UP | AI214272 |
| 653 | UP | AI216627 |
| 654 | UP | AI217765 |
| 655 | UP | AI219651 |
| 656 | UP | AI224908 |
| 657 | UP | AI239760 |
| 658 | UP | AI242117 |
| 659 | UP | AI242356 |
| 660 | UP | AI243374 |
| 661 | UP | AI243608 |
| 662 | UP | AI243663 |
| 663 | UP | AI247218 |
| 664 | UP | AI271617 |
| 665 | DOWN | AI272678 |
| 666 | UP | AI276564 |
| 667 | UP | AI279626 |
| 668 | UP | AI280437 |
| 669 | UP | AI286198 |
| 670 | UP | AI291262 |
| 671 | UP | AI298267 |
| 672 | UP | AI301973 |
| 673 | UP | AI309066 |
| 674 | UP | AI312671 |
| 675 | UP | AI347570 |
| 676 | UP | AI347657 |
| 677 | UP | AI349468 |
| 678 | DOWN | AI360323 |
| 679 | UP | H04313 |
| 680 | UP | H05970 |
| 681 | UP | H08785 |
| 682 | DOWN | H10641 |
| 683 | DOWN | H11658 |
| 684 | DOWN | H15452 |
| 685 | UP | H15559 |
| 686 | DOWN | H16238 |
| 687 | DOWN | H17800 |
| 688 | DOWN | H43101 |
| 689 | UP | H46966 |
| 690 | DOWN | H47121 |
| 691 | DOWN | H52198 |
| 692 | DOWN | H52531 |
| 693 | UP | H52546 |
| 694 | UP | H54253 |
| 695 | DOWN | H57947 |
| 696 | DOWN | H60408 |
| 697 | UP | H63248 |
| 698 | DOWN | H70895 |
| 699 | DOWN | H72866 |
| 700 | DOWN | H81000 |
| 701 | DOWN | H85107 |
| 702 | DOWN | H88540 |
| 703 | UP | H91700 |
| 704 | UP | H97413 |
| 705 | UP | H97987 |
| 706 | UP | N20862 |
| 707 | DOWN | N21630 |
| 708 | UP | N22796 |
| 709 | DOWN | N23897 |
| 710 | DOWN | N25338 |
| 711 | UP | N33229 |
| 712 | UP | N33555 |
| 713 | DOWN | N35046 |
| 714 | DOWN | N48689 |
| 715 | DOWN | N49276 |
| 716 | UP | N51336 |
| 717 | UP | N52627 |
| 718 | DOWN | N54855 |
| 719 | UP | N54899 |
| 720 | UP | N56860 |
| 721 | UP | N57955 |
| 722 | UP | N58081 |
| 723 | UP | N58543 |
| 724 | DOWN | N62378 |
| 725 | DOWN | N62404 |
| 726 | DOWN | N63490 |
| 727 | UP | N63906 |
| 728 | DOWN | N66102 |
| 729 | DOWN | N66104 |
| 730 | DOWN | N73278 |
| 731 | UP | N77929 |
| 732 | UP | N90849 |
| 733 | DOWN | R00311 |
| 734 | DOWN | R02083 |
| 735 | UP | R08356 |
| 736 | DOWN | R11341 |
| 737 | UP | R15104 |
| 738 | DOWN | R15891 |
| 739 | DOWN | R15979 |
| 740 | UP | R20625 |
| 741 | UP | R20640 |
| 742 | DOWN | R31272 |
| 743 | DOWN | R33616 |
| 744 | DOWN | R37780 |
| 745 | UP | R38660 |
| 746 | UP | R39951 |
| 747 | UP | R40123 |
| 748 | UP | R43555 |
| 749 | UP | R51627 |
| 750 | UP | R52531 |
| 751 | DOWN | R52541 |
| 752 | DOWN | R52650 |
| 753 | UP | R53235 |
| 754 | UP | R55784 |
| 755 | DOWN | R59072 |
| 756 | DOWN | R68794 |
| 757 | DOWN | R70402 |
| 758 | UP | R73637 |
| 759 | UP | R74206 |
| 760 | DOWN | R77434 |
| 761 | UP | R79952 |
| 762 | UP | R80259 |
| 763 | UP | R83878 |
| 764 | DOWN | R87758 |
| 765 | UP | R98791 |
| 766 | DOWN | R99110 |
| 767 | UP | T54672 |
| 768 | DOWN | T57803 |
| 769 | UP | T67181 |
| 770 | DOWN | T71889 |
| 771 | UP | T77897 |
| 772 | UP | T78450 |
| 773 | UP | T90962 |
| 774 | DOWN | T96375 |
| 775 | DOWN | T98542 |
| 776 | DOWN | T98927 |
| 777 | DOWN | T99029 |
| 778 | DOWN | W67951 |
| 779 | DOWN | W73883 |
| 780 | DOWN | W81432 |
| 781 | DOWN | W90624 |
| 782 | DOWN | W90749 |
| 783 | DOWN | W95876 |

TABLE 4
Most relevant gene transcripts

| SEQ ID NO | vailD | Accession | Refseq | Description |
|---|---|---|---|---|
| 378 | 25977 | AA903175 | NM_007360.1 | Homo sapiens killer cell lectin-like receptor subfamily K, member 1 (KLRK1), mRNA |
| 203 | 8411 | AA419251 | NM_003641 | Homo sapiens interferon induced transmembrane protein 1 (9-27) (IFITM1), mRNA |
| 448 | 13294 | AA431761 | NM_153051 | Homo sapiens myotubularin related protein 3 (MTMR3) transcript variant 2, mRNA |
| 352 | 9521 | R55763 | NM_020819 | Homo sapiens KIAA1411 (KIAA1411), mRNA |
| 576 | 33041 | AA928710 | | |
| 73 | 31521 | AI364359 | NM_007129.2 | Homo sapiens Zic family member 2 (odd-paired homolog, Drosophila) (ZIC2), mRNA |
| 759 | 13909 | R74206 | NM_080664 | Homo sapiens chromosome 14 open reading frame 126 (C14orf126), mRNA |
| 55 | 25057 | AA862473 | NM_020372.2 | Homo sapiens solute carrier family 22 (organic cation transporter), member 71 |
| | | | | (SLC22A17), transcript variant 1, mRNA |
| 94 | 28272 | R49756 | NM_003471.2 | Homo sapiens potassium voltage-gated channel, shaker-related subfamily, beta member 1 |
| | | | | (KCNAB1), transcript variant 2, mRNA |
| 162 | 31915 | AI671599 | NM_002556.2 | Homo sapiens oxysterol binding protein (OSBP), mRNA |
| 284 | 7647 | W32509 | NM_012121 | Homo sapiens CDC42 effector protein (Rho GTPase binding) 4 (CDC42EP4), mRNA |
| 108 | 5618 | N51651 | NM_015099 | Homo sapiens calmodulin binding transcription activator 2 (CAMTA2), mRNA |
| 52 | 11452 | AA446864 | NM_019000 | Homo sapiens hypothetical protein FLJ20152 (FLJ20152), mRNA |
| 155 | 17242 | AA703250 | NM_004697 | Homo sapiens PRP4 pre-mRNA processing factor 4 homolog (yeast) (PRPF4), mRNA |
| 150 | 39841 | AI203283 | NM_032726.2 | Homo sapiens phospholipase C, delta 4 (PLCD4), mRNA |
| 177 | 12032 | N35020 | NM_016422 | Homo sapiens ring finger protein 141 (RNF141), mRNA |
| 309 | 25587 | AA904604 | XM_096472.2 | PREDICTED: Homo sapiens similar to RIKEN cDNA 1700029I15 (LOC143678), mRNA |
| 601 | 26407 | AA975413 | | |
| 751 | 30082 | R52541 | | |
| 596 | 26696 | AA971493 | | |
| 156 | 18958 | N48345 | NM_173680 | Homo sapiens hypothetical protein MGC33584 (MGC33584), mRNA |
| 764 | 23764 | R87758 | | |
| 128 | 15927 | AA463461 | NM_024325 | Homo sapiens zinc finger protein 343 (ZNF343), mRNA |
| 704 | 32026 | H97413 | | |
| 663 | 38458 | AI247218 | | |
| 706 | 11108 | N20862 | NM_025133 | Homo sapiens F-box protein 11 (FBXO11), transcript variant 1, mRNA |
| 630 | 29950 | AI025349 | | |
| 101 | 16379 | R40567 | NM_004229 | Homo sapiens cofactor required for Sp1 transcriptional activation, subunit 2, 150 kDa |
| | | | | (CRSP2), mRNA |
| 123 | 5128 | N63635 | NM_002648 | Homo sapiens pim-1 oncogene (PIM1), mRNA |
| 429 | 12839 | AA136071 | NR_001569 | Homo sapiens a disintegrin and metalloproteinase domain 3a (cyritestin 1) (ADAM3A) on |
| | | | | chromosome 8 |
| 364 | 5756 | AA148683 | NM_001002860 | Homo sapiens BTB (POZ) domain containing 7 (BTBD7), transcript variant 1, mRNA |
| 100 | 20964 | AA780712 | NM_003588.2 | Homo sapiens cullin 4B (CUL4B), mRNA |
| 103 | 19816 | AA706969 | NM_017893 | Homo sapiens sema domain, immunoglobulin domain (Ig), transmembrane domain (TM) |
| | | | | and short cytoplasmic domain, (semaphorin) 4G (SEMA4G), mRNA |
| 315 | 13279 | AA429886 | XM_370965 | PREDICTED: Homo sapiens similar to hypothetical protein BC011981 (LOC388242), |
| | | | | mRNA |
| 259 | 7080 | AA055163 | NM_001232 | Homo sapiens calsequestrin 2 (cardiac muscle) (CASQ2), mRNA |
| 733 | 14265 | R00311 | XM_117117 | PREDICTED: Homo sapiens hypothetical gene FLJ13072 (FLJ13072), mRNA |
| 697 | 30308 | H63248 | | |
| 744 | 16786 | R37780 | NM_018133 | Homo sapiens ring finger protein 184 (RNF184), mRNA |
| 369 | 20594 | AA489033 | NM_001009894.2 | Homo sapiens hypothetical protein DKFZp434N2030 (DKFZp434N2030), mRNA |
| 765 | 28959 | R98791 | | |
| 423 | 7868 | AA041254 | NM_152417 | Homo sapiens hypothetical protein FLJ32370 (FLJ32370), mRNA |
| 130 | 31418 | AI276745 | NM_000956.2 | Homo sapiens prostaglandin E receptor 2 (subtype EP2), 53 kDa (PTGER2), mRNA |
| 673 | 39557 | AI309066 | | |
| 503 | 24470 | AA700433 | | |
| 553 | 25631 | AA907727 | | |
| 282 | 8423 | AA044390 | NM_001001521 | Homo sapiens UDP-glucose pyrophosphorylase 2 (UGP2), transcript variant 2, mRNA |
| 98 | 14686 | AA127221 | NM_003856 | Homo sapiens interleukin 1 receptor-like 1 (IL1RL1), transcript variant 2, mRNA |
| 741 | 23625 | R20640 | | |
| 534 | 25227 | AA884326 | | |
| 165 | 31449 | AI300766 | NM_003989.2 | Homo sapiens paired box gene 2 (PAX2), transcript variant d, mRNA |
| 637 | 36066 | AI126464 | | |
| 470 | 17370 | AA521339 | NM_002547 | Homo sapiens oligophrenin 1 (OPHN1), mRNA |
| 776 | 29018 | T98927 | | |
| 707 | 30502 | N21630 | | |
| 560 | 33241 | AA917044 | | |
| 709 | 29342 | N23897 | | |
| 572 | 25667 | AA927170 | | |
| 257 | 25006 | AA826373 | NM_205842.1 | Homo sapiens NCK-associated protein 1 (NCKAP1), transcript variant 2, mRNA |
| 229 | 21895 | AA670305 | XM_117294.6 | PREDICTED: Homo sapiens hypothetical protein LOC200933 (LOC200933), mRNA |
| 440 | 20742 | AA291138 | | |
| 373 | 22475 | AA703019 | NM_016530.2 | Homo sapiens RAB8B, member RAS oncogene family (RAB8B), mRNA |
| 763 | 36396 | R83878 | | |
| 235 | 20102 | AA626335 | NM_014567.2 | Homo sapiens breast cancer anti-estrogen resistance 1 (BCAR1), mRNA |
| 246 | 1455 | T70056 | NM_002940 | Homo sapiens ATP-binding cassette, sub-family E (OABP), member 1 (ABCE1), mRNA |
| 642 | 35753 | AI151218 | | |
| 42 | 9245 | AA291513 | NM_001707 | Homo sapiens B-cell CLL/lymphoma 7B (BCL7B), transcript variant 1, mRNA |
| 531 | 25235 | AA883127 | | |
| 86 | 16950 | AA916325 | NM_003739 | Homo sapiens aldo-keto reductase family 1, member C3 (3-alpha hydroxysteroid |
| | | | | dehydrogenase, type II) (AKR1C3), mRNA |
| 687 | 9560 | H17800 | NM_052857 | Homo sapiens coiled-coil domain containing 16 (CCDC16), mRNA |
| 683 | 5098 | H11658 | NM_012120 | Homo sapiens CD2-associated protein (CD2AP), mRNA |
| 669 | 38215 | AI286198 | | |
| 84 | 20085 | AA634427 | NM_018405.2 | Homo sapiens hypothetical protein, clone 2746033 (HSA272196), mRNA |
| 716 | 19053 | N51336 | NM_001004341 | Homo sapiens FLJ16478 protein (FLJ16478), mRNA |
| 297 | 21091 | AI014441 | NM_001003397.1 | Homo sapiens tumor protein D52-like 1 (TPD52L1), transcript variant 4, mRNA |
| 278 | 71 | AA479196 | NM_004128 | Homo sapiens general transcription factor IIF, polypeptide 2 (30 kD subunit) (GTF2F2), |
| | | | | mRNA |
| 17 | 27256 | AI017101 | NM_015001.2 | Homo sapiens spen homolog, transcriptional regulator (Drosophila) (SPEN), mRNA |
| 441 | 2221 | AA293192 | NM_002161 | Homo sapiens isoleucine-tRNA synthetase (IARS), transcript variant short, mRNA |
| 593 | 33594 | AA970505 | | |
| 473 | 23225 | AA609215 | | |
| 45 | 12056 | AA598780 | NM_014776 | Homo sapiens G protein-coupled receptor kinase interactor 2 (GIT2), transcript variant 3, |
| | | | | mRNA |
| 766 | 23801 | R99110 | | |
| 465 | 20810 | AA490225 | | |
| 221 | 15286 | AA479950 | NM_198531 | Homo sapiens ATPase, Class II, type 9B (ATP9B), mRNA |
| 181 | 1160 | AA454819 | NM_002746 | Homo sapiens mitogen-activated protein kinase 3 (MAPK3), mRNA |
| 573 | 26215 | AA927221 | | |
| 782 | 5759 | W90749 | NM_203372 | Homo sapiens acyl-CoA synthetase long-chain family member 3 (ACSL3), transcript |
| | | | | variant 2, mRNA |
| 779 | 12500 | W73883 | NM_016065 | Homo sapiens mitochondrial ribosomal protein S16 (MRPS16), nuclear gene encoding |
| | | | | mitochondrial protein, mRNA |
| 617 | 27321 | AA993998 | | |
| 387 | 1847 | AA487265 | NM_014752 | Homo sapiens signal peptidase complex subunit 2 homolog (S. cerevisiae) (SPCS2), |
| | | | | mRNA |
| 466 | 20571 | AA490892 | | |
| 167 | 32174 | AA101088 | NM_017846.3 | Homo sapiens tRNA selenocysteine associated protein (SECP43), mRNA |
| 262 | 8370 | N63943 | NM_000239 | Homo sapiens lysozyme (renal amyloidosis) (LYZ), mRNA |
| 197 | 21010 | AA903500 | NM_021809.4 | Homo sapiens TGFB-induced factor 2 (TALE family homeobox) (TGIF2), mRNA |
| 294 | 9556 | H23444 | NM_213609 | Homo sapiens family with sequence similarity 19 (chemokine (C-C motif)-like), |
| | | | | member A1 (FAM19A1), mRNA |
| 375 | 16510 | R51015 | NM_000369 | Homo sapiens thyroid stimulating hormone receptor (TSHR), transcript variant 1, mRNA |
| 175 | 17327 | AA670408 | NM_004048 | Homo sapiens beta-2-microglobulin (B2M), mRNA |
| 4 | 15278 | AA398356 | NM_020645 | Homo sapiens nuclear receptor interacting protein 3 (NRIP3), mRNA |
| 60 | 31846 | AI681015 | NM_004232.2 | Homo sapiens suppressor of cytokine signaling 6 (SOCS6), mRNA |
| 251 | 2910 | N55274 | NM_003239 | Homo sapiens transforming growth factor, beta 3 (TGFB3), mRNA |

### EXAMPLE 2

### Determination of Protein Levels in Blood

Among the biomarker's genes that are expressed differentially in asthma and non-asthma patients (namely, gene transcripts that constitute the “final asthma gene expression profile”), there are genes that encode known proteins. These known proteins are either secreted into the blood, localized intracellularly or localized in the cell membrane as transmembrane proteins (see Table 5). Detection of the proteins can be performed by protein detection methods known in the art such as methods based on antigen-antibody reactions including, but not limited to, enzyme-linked immunosorbent assay (ELISA), western blotting, protein arrays, antibody based biosensors, or by protein analysis methods such as mass spectrometry.

Thus, proteins encoded by gene transcripts that are identified as biomarkers, can be quantified in blood samples or in isolated blood cells by different protein detection methods, and hence their detection can enable diagnosis, prognosis and monitoring of the disease.

TABLE 5
Examples of genes with extra or intracellular known localization.

| SEQ. ID | | | | |
|---|---|---|---|---|
| NO. | Localiztion | Accession | Description | Regulation |
| 294 | Secreted | NM_213609 | FAM19A1 family with sequence | Down |
| | | | similarity 19 (chemokine (C-C | |
| | | | motif)-like), member A1 | |
| 251 | Secreted | NM_003239 | TGFB3 transforming growth factor, | UP |
| | | | beta 3 | |
| 175 | secreted | NM_004048 | B2M beta-2-microglobulin | UP |
| 203 | Transmembrane | NM_003641 | IFITM1 interferon induced | Down |
| | | | transmembrane protein 1 (9-27) | |
| 375 | Transmembrane | NM_000369 | TSHR thyroid stimulating hormone | UP |
| | | | receptor | |
| 94 | Transmembrane | NM_003471 | KCNAB1 potassium voltage-gated | UP |
| | | | channel, shaker-related subfamily, | |
| | | | beta member 1 | |
| 197 | Cellular | NM_021809 | TGIF2 TGFB-induced factor 2 | UP |
| | | | (TALE family homeobox) | |
| 4 | Cellular | NM_020645 | NRIP3 nuclear receptor interacting | UP |
| | | | protein 3 | |
| 60 | Cellular | NM_004232 | SOCS6 suppressor of cytokine | UP |
| | | | signaling 6 | |

It will be appreciated by persons skilled in the art that the present invention is not limited by what has been particularly shown and described herein above. Rather the scope of the invention is defined by the claims that follow.

What is claimed is:

1. A method of identifying at least one biomarker for a disease comprising the steps of:
a) determining the level of at least one gene transcript in a subpopulation of blood cells obtained from at least one subject having the disease, the at least one subject having the disease being a member of a closed population; and
b) comparing the level of the at least one gene transcript from step a) with the level of said at least one gene transcript in the subpopulation of blood cells obtained from at least one subject not having the disease, the at least one subject not having the disease being a member of the closed population, wherein a gene transcript which displays differing levels in the comparison of step b) is identified as being a biomarker for said disease.

2. The method according to claim 1 further comprising the step of:
c) determining the level of the at least one gene transcript in said subpopulation of blood cells obtained from at least one subject having the disease, the at least one subject having the disease being a member of an open population; and
d) comparing the level of the at least one gene transcript from step c) with the level of said at least one gene transcript in the subpopulation of blood cells obtained from at least one subject not having the disease, the at least one subject not having the disease being a member of an open population, wherein a gene transcript which displays corresponding changes in levels of gene expression in the comparisons of steps b) and d) is identified as being a biomarker for said disease.

3. The method according to claim 2, wherein the corresponding changes in levels of gene expression are increasing levels.

4. The method according to claim 2, wherein the corresponding changes in levels of gene expression are decreasing levels.

5. The method according to claim 1, wherein the at least one biomarker is a plurality of biomarkers.

6. The method according to claim 5, wherein the plurality of biomarkers comprises at least 100 biomarkers.

7. The method according to claim 5, wherein the plurality of biomarkers comprises at least 500 biomarkers.

8. The method according to claim 1, wherein the subpopulation of blood cells is peripheral white blood cells.

9. The method according to claim 8, wherein the subpopulation of blood cells is selected from the group consisting of monocytes, lymphocytes, neutrophils, eosinophils, and basophils.

10. The method according to claim 1, wherein the disease is selected from the group consisting of cardiovascular disorders, immune diseases, muscular diseases, mood diseases, autoimmune diseases, respiratory diseases, endocrine disorders, neurological disorders, metabolic disorders and cellular proliferative disorders.

11. The method according to claim 10, wherein the respiratory disease is asthma.

12. The method according to claim 11, wherein the biomarker is selected from the group consisting of SEQ ID NOs: 1-783 and complements thereof.

13. The method according to claim 1, wherein the step of determining the level of at least one gene transcript comprises microarray hybridization.

14. The method according to claim 13, wherein the microarray hybridization comprises hybridizing a first plurality of isolated nucleic acid molecules to an array comprising a second plurality of isolated nucleic acid molecules.

15. The method according to claim 14, wherein the first plurality of isolated nucleic acid molecules is selected from the group consisting of RNA, DNA, cDNA, and PCR products.

16. The method according to claim 14, wherein the second plurality of isolated nucleic acid molecules is selected from the group consisting of RNA, DNA, cDNA, PCR products, oligonucleotides and ESTs.

17. The method according to claim 14, wherein the array comprises one or more of the identified biomarkers.

18. The method according to claim 14, wherein the array comprises a plurality of isolated nucleic acid molecules corresponding to one or more of the identified biomarkers or complements thereof.

19. A method of diagnosing, monitoring or prognosing a disease in a subject comprising the steps of:
a) determining the level of at least one gene transcript in a subpopulation of blood cells obtained from the subject, wherein the at least one gene transcript corresponds to a biomarker, the biomarker having been determined according to claim 1; and
b) comparing the level of said at least one gene transcript of step a) with the level of said at least one gene transcript in a reference gene transcript profile, thereby determining the status of the disease in said subject.

20. The method according to claim 19, wherein the step of determining the level of the at least one gene transcript comprises determining the expression level of the gene.

21. The method according to claim 19, wherein the step of determining the level of the at least one gene transcript comprises determining the level of the polypeptide gene transcript.

22. The method according to claim 19, wherein the subpopulation of blood cells is peripheral white blood cells.

23. The method according to claim 22, wherein the subpopulation of blood cells is selected from the group consisting of lymphocytes, monocytes, neutrophils, eosinophils and basophils.

24. The method according to claim 19, wherein the biomarker is a plurality of biomarkers.

25. The method according to claim 19, wherein the disease is selected from the group consisting of cardiovascular disorders, immune diseases, muscular diseases, mood disorders, autoimmune diseases, respiratory diseases, endocrine disorders, neurological disorders, metabolic disorders and cellular proliferative disorders.

26. The method according to claim 25, wherein the respiratory disease is asthma.

27. The method according to claim 26, wherein the biomarkers are selected from the group consisting of SEQ ID NOs: 1-783 and complements thereof.

28. The method according to claim 19, wherein the step of determining the level of at least one gene transcript comprises microarray hybridization.

29. The method according to claim 28, wherein the microarray hybridization comprises hybridizing a first plurality of isolated nucleic acid molecules to an array comprising a second plurality of isolated nucleic acid molecules.

30. The method according to claim 29, wherein the first plurality of isolated nucleic acid molecules is selected from the group consisting of RNA, DNA, cDNA and PCR products.

31. The method according to claim 19, wherein the second plurality of isolated nucleic acid molecules is selected from the group consisting of RNA, DNA, cDNA, PCR products, oligonucleotides and ESTs.

32. The method according to claim 19, wherein the array comprises one or more of the identified biomarkers.

33. The method according to claim 19, wherein the array comprises a plurality of isolated nucleic acid molecules corresponding to one or more of the identified biomarkers or complements thereof.

34. A plurality of isolated nucleic acid molecules corresponding to one or more biomarkers or complements thereof, the biomarkers having been identified according to claim 1.

35. The plurality of isolated nucleic acid molecules according to claim 34, wherein the biomarkers being biomarkers for asthma.

36. The plurality of isolated nucleic acid molecules according to claim 35, wherein the biomarkers of asthma are selected from the group consisting of SEQ ID NOs:1-783 or complements thereof.

37. An array comprising the plurality of isolated nucleic acid molecules according to claim 34.
